(19) United States
(12) Patent Application Publication
(10) Pub. No.: US 2021/0096127 A1
(43) Pub. Date: Apr. 1, 2021

(54) MEANS AND METHODS FOR MONITORING SCAR DEVELOPMENT

(71) Applicant: HELMHOLTZ ZENTRUM MÜNCHEN - DEUTSCHES FORSCHUNGSZENTRUM FÜR GESUNDHEIT UND UMWELT (GMBH), Neuherberg (DE)

(72) Inventors: Yuval RINKEVICH, Munich (DE); Dongsheng JIANG, Ingolstadt (DE); Donovan CORREA-GALLEGOS, Munich (DE)

(21) Appl. No.: 16/968,326

(22) PCT Filed: Aug. 2, 2019
(86) PCT No.: PCT/EP2019/053089

§ 371 Date: Aug. 7, 2020

(30) Foreign Application Priority Data
Feb. 9, 2018 (EP) 18155916.2
Feb. 12, 2018 (LU) LU100695

(51) Int. Cl. G01N 33/50; C12N 5/071

(52) U.S. Cl. CPC G01N 33/5088; G01N 2333/78; C12N 5/0698

(57) ABSTRACT

The present invention relates to a method for generating an ex vivo skin sample being capable of developing scar. Further, the present invention comprises a method for screening for a compound which modulates scar development. Additionally, a preparation comprising a scarred full thickness skin sample obtainable by the method of the present invention is also envisaged. Finally, the present invention also encompasses a preparation comprising a full thickness skin model comprising a full thickness skin sample immersed and unethered in liquid culture.

### TECHNICAL FIELD OF THE INVENTION

The present invention relates to a method for generating an ex vivo skin sample being capable of developing scar. Further, the present invention comprises a method for screening for a compound which modulates scar development. Additionally, a preparation comprising a scarred full thickness skin sample obtainable by the method of the present invention is also envisaged. Finally, the present invention also encompasses a preparation comprising a full thickness skin model comprising a full thickness skin sample immersed and unethered in liquid culture.

### BACKGROUND ART

‘Skin scarring’ groups a range of pathologies that occur in response to dermatologic injuries, syndromes and diseases. Each year in the developed world 100 million patients are diagnosed with scars that result from surgical procedures alone. There are an estimated 11 million keloid scars and four million burn scars, 70% of which occur in children. These figures include only clinical cases, so the overall global incidence of scarring is doubtless much higher. People with abnormal skin scarring may face physical, aesthetic, psychological, and social consequences that may be associated with substantial emotional and financial costs.

Overall, scars are an enormous biomedical, clinical and cosmetic problem and surprisingly it is little known about how dermal scars develop at the sites of skin injuries. Current treatment options to eradicate or prevent scarring in humans and domestic animals are unreliable and there are no prescription drugs for the prevention or treatment of dermal scarring.

So far, our knowledge on mammalian scar tissue development stems from snap shot images from histologic slides of this patho-biological skin event or from in-vitro assays that study either fibroblast migrations, proliferation or secretions which are artificial environments both biochemically and biophysically. These capture only minimal aspects of a complex, multifactorial and dynamic process. Thus, mammalian scar tissue development has remained obscure and no existing model explains mechanistically how scars are created. Key steps/hallmarks of mammalian scar development, and the dynamics of its morphological and cellular events have largely remained obscure.

In mammalian skin, technical considerations such as tissue penetration, image resolution and organ movements, limit our observations into this dynamic process to snap shot histological images (in adult rodents, scar develops after 10-14 days post injury) imaging of sub-epidermal development extremely challenging. Indeed, mammalian scars usually develop only from a certain depth of injury and develop gradually over several days. Both depth and time frame hinders the imaging of in vivo mammalian wounds, limiting the analysis to snap shot histological images.

In light of such limitations several models have been used that mimic distinct biological processes of fibroblasts, but with inherent limitations. In-silico (Zaritsky et al., 2015), or in-vitro wound healing assays employ either fibroblastic cell lines, primary fibroblasts from wounded skin tissues (from animal models and human subjects), or from keloid or hypertrophic scars (human subjects) grown on two dimension (2D) tissue culture substrates. While these assays aim to recapitulate fibroblast activity in an injured tissue, we would argue they in fact mask cellular and molecular dynamics native to a physiologic tissue environment. Indeed, we have shown cultured fibroblasts loose their transcriptional, proteomic and surface marker expressions. Further, 2D tissue culture assays lack hallmarks of scar tissue development that evolve only in-vivo and in 3D.

Three dimension (3D) culture assays such as tissue engineered skin substitutes employ artificial substrates infused with fibroblast cell-lines that fail to mimic a physiologic extracellular tissue environment. While these assays allow modeling the interactions between fibroblasts and ECM in a controlled manner, thus recapitulating some aspects of the injury cascade, they lack the physiologic properties and cellular complexity. Critically, the above assays lack the cellular complexity native to a tissue including the scar's fibroblastic cells-of-origin (Rinkevich et al., 2015, Science 348 (6232)) precluding any interpretation into bona fide pathomechanisms of scar develop.

Thus, there still exists a need for a skin tissue model that recapitulates dermal scar tissue development as in vivo and that enables the analysis of scar formation.

Thus, the objective of the present invention is to comply with this need.

The solution of the present invention is described in the following, exemplified in the appended examples, illustrated in the figures and reflected in the claims.

### SUMMARY OF THE INVENTION

The present invention comprises a skin tissue model that recapitulates dermal scar tissue development as in vivo (termed Scar-in-a-Dish; SCAD). Skin biopsies as small as about 2 mm from back skin that maintain the multifactorial characteristics and cellular complexity native to physiologic skin generate authentic dermal scars. Using fibroblast lineage specific Cre murine drivers in combination with fluorescence and multi-color reporters for clonal cell tracing, it is shown that SCAD develops from their authentic cells-of-origin as in vivo. SCAD development shows reduced phenotypic variability as compared to in vivo wound healing models.

The present invention comprises a method for generating an ex vivo skin sample being capable of developing scar, comprising a) culturing a full thickness skin sample immersed and untethered in liquid culture; b) determining whether a scar is developed by the full thickness skin sample in step (a); and c) obtaining a scarred full thickness skin sample, wherein the full thickness skin sample comprises fascia.

Additionally, the present invention may also comprise the method of the present invention as mentioned above, wherein the full thickness skin sample may further comprise epidermis, dermis, subcutis. Additionally, the full thickness skin sample may be obtained from a mammal. Preferably, the full thickness skin sample is a punch biopsy. In a preferred embodiment, the punch biopsy is from a dorsal region. The full thickness skin sample may also be a fresh sample. Preferably, the full thickness skin sample has an average thickness of about 1 to 3 mm.

Preferably, the mammal is a mouse or human for a method for generating an ex vivo skin sample. The mouse may be in a developmental fetal stage of at least E18.5 up to neonatal stage P10.

The present invention may provide a method for generating an ex vivo skin sample being capable of developing a scar, wherein the liquid culture is suspension culture.

The full thickness skin sample may be cultured for at least 4 days. Culturing may be performed by using a DMEM/F-12 medium comprising 10% FBS, 1× GlutaMAX, 1× Penicillin/streptomycin, and 1× MEM non-essential amino acids.

The present invention may encompass a method for generating an ex vivo skin sample, comprising determining whether the full thickness skin sample contains cells expressing CK14, Engrailed-1, CD26, N-Cadherin, alpha-smooth muscle actin (α-SMA), fibroblast specific proteins 1 (FSP1) and/or platelet derived growth factor receptors alpha (PDGFRα) and beta (PDGFR).

Also comprised herein is a method for generating an ex vivo skin sample comprising determining whether the full thickness skin sample contains cells expressing α-SMA, CD90, ER-TR7, PDGFRα, Sca1, βIIITubulin, CD31, MOMA-2, F4/80, CD24, CD34, CD26, Dlk1, Fn1, Col14a1, Emilin2, Gsn and/or Nov.

Further, the present invention may comprise a method for generating an ex vivo skin sample, wherein in step (b) determining whether a scar is developed by the full thickness skin sample in step (a) is done by visual inspection.

Additionally, the present invention may envisage a method for generating an ex vivo skin sample, wherein in step (b) determining whether a scar is developed by the full thickness skin sample in step (a) comprises determining whether collagen type I, collagen type III and/or fibronectin is present in said full thickness skin sample.

The present invention encompasses a method for screening for a compound which modulates scar development, comprising a) carrying out the method mentioned above in the presence of a compound of interest; and b) determining whether said compound of interest modulates scar development in comparison to carrying out the method mentioned above in the absence of said compound of interest. Modulation of scar development may be inhibition of scar development or promotion of scar development.

The present invention comprises a preparation comprising a scarred full thickness skin sample obtainable by the method for generating an ex vivo skin sample as mentioned above.

Further, the present invention envisages a preparation comprising a full thickness skin model comprising a full thickness skin sample immersed and untethered in liquid culture, wherein the full thickness skin sample comprises fascia.

Also comprised herein is a preparation comprising a full thickness skin model comprising a full thickness skin sample immersed and untethered in liquid culture, wherein the full thickness skin sample further comprises epidermis, dermis, subcutis. The present invention may comprise a preparation comprising a full thickness skin model comprising a full thickness skin sample, wherein the full thickness skin sample is obtained from a mammal.

Also comprised by the present invention may be a preparation comprising a full thickness skin model comprising a full thickness skin sample, wherein the full thickness skin sample is a punch biopsy.

The present invention may envisage a preparation comprising a full thickness skin model comprising a full thickness skin sample, wherein the punch biopsy is from a dorsal region.

The present invention may encompass a preparation comprising a full thickness skin model comprising a full thickness skin sample, wherein the full thickness skin sample is a fresh sample.

Additionally, the present invention may envisage a preparation comprising a full thickness skin model comprising a full thickness skin sample, wherein the mammal is a mouse or human.

Further, the present invention may envisage a preparation comprising a full thickness skin model comprising a full thickness skin sample, wherein the mouse is in a developmental fetal stage of at least E18.5 up to neonatal stage P10.

The present invention may envisage a preparation comprising a full thickness skin model comprising a full thickness skin sample, wherein the full thickness skin sample has an average thickness of about 1 to 3 mm.

Also comprised by the present invention may be a preparation comprising a full thickness skin model comprising a full thickness skin sample for use in a method for screening for a compound which modulates scar development.

Additionally, the present invention may envisage a preparation comprising a full thickness skin model comprising a full thickness skin sample for use in therapy or diagnosis.

### BRIEF DESCRIPTION OF THE FIGURES

FIG. 1: Development of scar Day 1-Day 5 observed with SCAD from newborn mice back-skin.

A) Whole-mount bright-field images of fresh (day 0) SCAD (D0) and SCADs 1 day (D1)-5 days (D5) after culture. Over time, SCAD tissues bent towards their ventral side. In some SCAD tissues lateral folds came into direct contact on their ventral sides, most likely the outcome of scar contractures that were generated from the tissue's centre (D5′). B) Masson's trichrome staining of cryosections from D0 to D5 SCAD. Cytoplasm and muscle fibers stained red, whereas collagen displays blue coloration. Nuclei have been stained black. Hair follicles are observed as dark oval pits. C) Enlarged pictures of the scar centres of SCAD as shown in (B).

FIG. 2: SCAD assay in 384-well plate.

A), B), C), D), E) D5 SCADs media treated only and grown in 384-well plate. F) D5 scar grown in 96-well plate as a control. G) and H) whole-mount images of 384-well plate grown SCADs. The collagen is stained blue and the red color represents the muscle fibres and cytoplasm. Nuclei have been stained black. Hair follicles are observed as dark oval pits.

FIG. 3: Human SCAD development from Day 1 until Day 10 in DMEM growth medium.

Day 1 (A), Day 2 (B), Day 3 (C), Day 4 (D), Day 5 (E), Day 7 (F), Day 10 (G), Day 10 (H) highlights tissue complexity. Migration of keratinocytes was observed around the SCAD epithelium.

FIG. 4: Migration of keratinocytes in Human Day 1 to Day 10 SCADs in DMEM growth medium.

Day 1 (A), Day 2 (B), Day 3 (C), Day 4 (D), Day 7 (E), Day 10 (F). A gradual movement of keratinocytes was observed with time progression. The yellow arrow indicates the position of marginal keratinocytes.

FIG. 5: Deposition of matrix fibres in D4 SCAD.

Representative 3D rendering images of D4 SCAD with immunolabelling of fibronectin (red), collagen I (magenta), and elastin (green). The lower panel shows the enlarged scar centres indicated by white squares in the upper panel. Scale bars in he upper panel=100 μm, scale bars in the lower panel=30 μm.

FIG. 6: Marker expression in SCAD.

A) Cryosections of day 5 SCAD samples were prepared from wild type neonatal mouse back-skin, and immunolabeled with CK14 (green) and CD26 (magenta). The nuclei are counterstained with DAPI. B) 3D rendering image of the scar center of a day 5 SCAD prepared from wild type neonatal mouse back-skin and immunolabeled with α-SMA (green). C) 3D rendering image of day 5 SCAD prepared from En1^{Cre};R26^{mTmG} neonatal mouse back-skin and immunolabeled with N-Cadherin (magenta).

FIG. 7: Chemical screening of SCAD with chemical Prestw-229.

A) Chemical structure of the positive control Prestw-229 (Nefopam hydrochloride), which is known to reduce scar formation. B), C), D) whole-mount image of D5 SCAD treated with Nefopam hydrochloride. E) and F) section of D5 SCAD treated with Nefopam hydrochloride and development of scar. The box in (E) highlights scar development under chemical influence. G) and H) section of control (media treated) D5 SCAD.

FIG. 8: Wound bed fibroblasts originate from the fascia.

A) Experimental approach to label fascial cells using Adeno-CMV-eGFP viral particles in neonates. B) Histology images of wound beds showing transduced eGFP⁺ (green) cells at dpw 3 (left) and 7 (right) co-stained with DAPI (blue). C) Experimental design to label fascia cells in adult mice using Dil dye. D) Histology images of wound beds showing Dil⁺ (red) cells at dpw 9 (left) and 14 (right) co-stained with DAPI (blue). E) Quantification of the Dil⁺ cells percentage from the total cells in the wound bed. N=55 and 53 sections analyzed from 5 biological replicates. F) Schematic description of chimeric skin transplantations in adult immunodeficient mice to determine the cellular contribution of dermis and fascia to the wound bed fibroblasts. The muscle+fascia from GFP⁺ (R26^{VT2/GK3}) back-skin biopsies was manually separated, as well as the epidermis+dermis from TdTomato⁺ (R26^{mTmG}) samples. Both fragments were mounted on the correct orientation to generate the chimeric grafts and a “wound” was punched through both segments in the middle of the graft before transplanting into the back of immunodeficient mice. Epi=epidermis, Der=dermis, Pc=Panniculus carnosus, Fas=fascia, TdTom=TdTomato. G) Histology images of wound beds showing epidermal+dermal-derived TdTomato⁺ cells (red) and fascia+muscle-derived GFP⁺ cells (green) at dpw 14. H) Quantification of the TdTomato⁺ or GFP⁺ cells percentage from the total labeled cells (TdTomato⁺+GFP⁺) in the wound bed and the wound margin. N=26 sections analyzed from 4 biological replicates. One-way ANOVA, multiple comparison Tukey test, confidence interval=95%. I) Representation of the image analysis pipeline. The epidermis from the red channel was digitally excluded and the wound bed and wound margin areas traced. The remaining dermal cells and fascial cells from the red and green channel respectively were independently analyzed. Dotted lines delimitate the wound bed. Arrowheads indicate the original injury site. Scale bars=200 microns. wb=wound bed.

FIG. 9: Fibroblasts/mesenchymal and cell type-specific marker expression in Dil-labeled cells.

Immunolabeling (green) of wound beds with Dil-labeled fascial cells (red) against fibroblasts/mesenchymal markers αSMA (A), CD29 (C), CD90 (E), ER-TR7 (G), Pdgfra (I), or Sca1 (K), and the specific markers for nerves (βIIITubulin, M), endothelial (CD31, O), lymphatics (Lyve1, Q), or macrophages (MOMA-2, S); co-stained with DAPI (blue). Quantification of the Dil⁺ cells percentage from the total cells in the wound bed expressing fibroblasts/mesenchymal markers αSMA (B), CD29 (D), CD90 (F), ER-TR7 (H), Pdgfra (J), or Sca1 (L), and the specific markers for nerves (βIIITubulin, N), endothelial (CD31, P), lymphatics (Lyve1, R), or macrophages (MOMA-2, T); at dpw 9 and 14. N=images analyzed from 5 biological replicates. Dotted lines delimitate the wound bed. Scale bars=200 microns.

FIG. 10: Cell type-specific marker expression in chimeric skin experiments.

Immunolabeling (magenta) of wound beds from fascia (green) dermis (red) chimeric grafts against specific markers for fibroblasts (αSMA, A), nerves (βIIITubulin, C), endothelial (CD31, E), lymphatics (Lyve1, I), or macrophages (F4/80 and MOMA-2, G and K). Quantification of the fascia-derived (GFP⁺) or dermis-derived (TdTomato⁺) cells fraction from the total labeled cells (GFP⁺, TdTomato⁺, and Marker⁺) expressing the specific markers for fibroblasts (αSMA, B), nerves (βIIITubulin, D), endothelial (CD31, F), lymphatics (Lyve1, J), or macrophages (F4/80 and MOMA-2, H and L). N=images analyzed from 4 biological replicates. Unpaired two-tailed T-test, confidence interval=95%. Dotted lines delimitate the wound bed. Scale bars=200 microns.

FIG. 11: Fascial-EPFs invasion into the wound bed dictates scar severity.

A) Schematic description of dermal- or fascial-EPFs labeling using chimeric skin grafts. Cre⁺ En1^{Cre};R26^{mTmG} back-skin samples were separated as before, and combined with samples of Cre⁻ littermates to only preserve the GFP⁺ cells (EPFs) in the fascia or dermis fragments. Deep injuries were performed by punching through the entire chimeric skin as before, while in superficial injuries only the epidermis+dermis was punched before mounting on the muscle+fascia part. At dpw 5 and 7, two pulses of EdU were administered. B)-H) Quantification of the fascial- and dermal-EPF fraction expressing CD24 (B), CD26 (C), Dlk1 (D), CD34 (E), Sca1 (F), CD29 (G) or CD9 (H) from the total EPFs (GFP⁺) in the wound bed, fascia, and dermis regions. N=sections analyzed from 5 biological replicates. One-way ANOVA, multiple comparison Tukey test, confidence interval=95%. wb=wound bed.

FIG. 12: Analysis of fibroblast clusters by digitally sorting.

After having digitally-sorted 5146 fibroblasts from 11 out of original 30 clusters, 3 clusters were enriched in the fascia. Dot plot showing the expression of principal shared markers present in fascial fibroblasts clusters. Fascial-fibroblasts clusters were enriched in the hypodermal marker Sca1 (Ly6a), and in pro-fibrotic markers such as CD26, fibronectin (Fn1) and Col14a1.

### DETAILED DESCRIPTION OF THE INVENTION

### Method

The present invention provides a method for generating an ex vivo skin sample being capable of developing scar, comprising a) culturing a full thickness skin sample immersed and untethered in liquid culture; b) determining whether a scar is developed by the full thickness skin sample in step (a); and c) obtaining a scarred full thickness skin sample.

For that, skin tissue from a mammal, in particular from mouse strains or humans as described elsewhere herein are collected, washed twice with cold DMEM/F12 medium to remove contaminating blood and also washed once with Hank's Balanced Salt Solution. Then, non-skin tissue at the ventral side of the skin tissue is removed and then the full thickness skin sample is created with a biopsy puncher. Additionally, the biopsy punch is cultured immersed and untethered in liquid culture, thereby obtaining a scarred full thickness skin sample.

In general, scars are common pathologic manifestations to skin injuries, dermatologic syndromes and diseases. Clinically speaking, skin scars cover a wide spectrum of phenotypes from thin-line scars to abnormal widespread, atrophic, hypertrophic, and keloid scars and scar contractures. For example, atrophic scars commonly arise after acne or chickenpox, while stretch marks (abdominal striae) that develop after pregnancy or weight gain are both versions of dermal scars in which the epidermis is unbreached.

Scars are tissue architectures that develop from fibroblastic cells and their extracellular matrix depositions of altered dermal/stromal tissue architectures. Indeed, scar exhibit tissue diversities with patterns that are stereotypic to the anatomic locations from which they arise. Instead of a porous ‘basket-weave’ scaffold that often develops, an impaired orientation of fiber alignment occurs that leads to severe pathologic manifestations such as reduced tensile strength and extensibility, loss of cellular composition, reduced function and growth, and structural weakness to the affected tissues and organs.

In this context, the “full thickness skin sample” refers to a skin sample comprising all skin layers including epidermis, dermis and subcutis (which comprises subcutaneous fat). Preferably such a skin sample comprising epidermis, dermis and subcutis further comprises fascia. A full-thickness skin sample including epidermis, dermis and subcutis is important for holding the newly developed scar. Fibroblasts in the dermal region need to be secluded between epidermis and subcutaneous fat tissue (humans) or thin muscle tissue (panniculus carnosus muscle in mice), so that they do not attach to the dish and migrate out of dermis. The inventors have also surprisingly found out as an alternative that the full thickness skin sample being used in the present invention should comprise fascia in order to generate an ex vivo skin sample being capable of developing scar. It was found that fascia is the major cellular source for all the cell types present in the wound bed and which dictate scar formation (illustrated in FIG. 8). The gelatinous connective tissue deep below the skin is known as superficial fascia.

Accordingly, for the purpose of the present invention, it is preferred that the term “full thickness skin sample” is a skin sample comprising fascia. Preferably, it may further comprise epidermis, dermis and subcutis. Thus, when referred to a full thickness skin sample being used in the present invention for the methods or the preparations of the present invention, it is preferred that the “full thickness skin sample” comprises fascia. Preferably, said full thickness skin sample which comprises fascia, further comprises epidermis, dermis and subcutis. For avoidance of doubt, the preferred full thickness skin sample in the context of the present invention is a skin sample comprising fascia, preferably further comprising epidermis, dermis and subcutis.

The term “immersed” refers to being submerged in liquid culture, meaning that the skin sample is not floating at the liquid air surface, instead is completely covered with liquid culture (medium). The skin sample is submerged into the culture medium, preferably with gentle touch of surgical thumb forceps (tweezers), thereby letting sink the skin sample to the bottom automatically by gravity. Shaking/stirring is not required, but may be performed as well.

Thus, the skin sample is completely surrounded by the liquid culture, sitting at the bottom of the culture plate, but without attachment to the culture plate.

To prevent the drying of keratinocytes on the epidermal surface, the biopsies are immersed into the media as described above to assure perfectly scar development.

Keratinocytes, a major cellular component of the epidermis, are responsible for restoring the epidermis after injury through a process termed epithelialization. A marker of keratinocytes is cytokeratin 14 (CK14).

Epithelialization is an essential component of wound healing used as a defining parameter of successful wound closure. A wound cannot be considered healed in the absence of re-epithelialization. The epithelialization process is impaired in all types of chronic wounds. To close the defect in the epidermis, keratinocytes at the wound edge must first loosen their adhesion to each other and to the basal lamina, and need to develop the flexibility to support migration over the freshly deposited matrix. This process is modulated sequentially beginning with disassembly of cell-cell and cell-substratum contacts maintained through desmosomes and hemidesmosomes, respectively. This release allows keratinocytes to start migrating from the wound edge over the denuded area, whereas keratinocytes behind the migrating tongue begin to proliferate. The process of keratinocyte migration thus plays an important and essential role in wound healing and scar development. Therefore, it was expected that in the SCAD model, migration of keratinocytes being marked with CK14 must be observed to successfully establish it as a working system.

The epidermis (mainly keratinocytes) is an important component of skin, thus believing keratinocytes need to be present in the system to produce the scar that is observed. Keratinocytes may have cross-talk with fibroblasts and regulate fibroblasts behaviour, why a “full-thickness” skin biopsy (including all skin layers) in the system may be used. In the SCAD system, not only keratinocytes and fibroblasts are present, but also all other cellular components of skin including, but not limited to leukocytes, endothelial cells etc. may be present.

The term “untethered” refers to unattached or unbound, in this context meaning that the skin sample does not attach (is not bound) to the bottom of the well, multi-well plate or even single culture dish or bag, where the sample is cultured in liquid culture in. By sitting at the bottom of the well/dish/bag, the sample does not attach to the bottom, because the fibroblasts are being present in the dermis and thus will not attach to the bottom of the well/dish/bag, as long as there is epidermis on one side and subcutaneous fat (or thin muscle tissue in case of mice) on the other side of the dermis. Thus, the unattached sample moves freely, if shaking the well, multi-well plate, single culture dish or the bag.

The term “liquid culture” in this context as used herein, refers to a specific medium (see also Example 2) being liquid/fluid, wherein the skin sample of the present invention may be cultured immersed and untethered in.

These conditions of culturing the skin sample of the present invention as mentioned herein, results in the development of scar formation.

Additionally, the full thickness skin sample may further comprise epidermis, dermis and subcutis.

In general, mammalian skin is composed of two layers: 1.) the epidermis (the outermost layer of the skin, an epithelial tissue) which serves as a barrier to infection, and 2.) the dermis. The epidermis can be further subdivided into the following layers: Stratum corneum, Stratum lucidum, Stratum granulosum, Stratum spinosum, Stratum basale. It constituets to approximately 95% of keratinocytes (McGrath et al., 2004, Rook's Textbook of Dermatology (7th ed.). Blackwell Publishing. pp. 3.1-3.6). Keratinocytess differentiate and delaminate from the basement membrane migrating upwards through the layers and, after losing the nucleus, fuse to squamous sheets. The production of keratinocytes is directly proportional to the loss of skin cells via shedding from the skin surface. Typical human primary keratinocytes possess an in vitro lifespan of around 15-20 population doublings (PDs) in serum-free and chemically defined media (Stoppler et al., 1997; Kiyono et al., 1998). To sum it up, the epidermis layer is important to keep the integrity of the biopsy. When the epidermis is removed by f.e. Dispase treatment, the tissue may dissociate much faster in culture.

The dermis provides tensile strength and elasticity to the skin by an extracellular matrix. Said ECM is composed of collagen fibrils, microfibrils, and elastic fibers, embedded in hyaluronan and proteoglycans (Breitkreutz et al., 2009, Histochemistry and cell biology. 132 (1): 1-10). Further, the dermis is composed of three major types of cells: fibroblasts, macrophages, and adipocytes. Together, the epidermis and the dermis form the cutis of the skin.

The subcutis (subcutaneous tissue) functions as a support and contains blood vessels and nerves for the above-mentioned skin layers. It further contains subcutaneous fat and also functions as fat storage.

Preferably, the full thickness skin sample comprises fascia. It may further comprise epidermis, dermis and subcutaneous fat tissue. In mice there is also panniculus carnosus muscle involved in the skin layers of the skin sample, which is part of the subcutaneous tissue, in particular a layer of striated muscle deep to the panniculus adiposus (the fatty layer of the subcutaneous tissue, also called subcutaneous fat). Human skin does not have a detectable panniculus carnosus muscle.

Preferably, the full thickness skin sample comprises fascia in mice. Said full thickness skin sample in mice may further comprise epidermis, dermis, subcutaneous fat and panniculus carnosus muscle. For the development of scar tissue and due to the thinness of neonatal skin making it impossible to remove particular layers of the skin sample without damaging the tissue architecture of the sample, it is of absolute relevance to have all the layers of the skin sample to develop scar tissue.

The full thickness skin sample may further be obtained from a mammal. Particularly, a mammal may be a rodent. A mammal may further be a rabbit, a mouse, a rat, a Guinea pig, a hamster, a dog, a cat, a pig, a cow, a goat, a sheep, a horse, a monkey, an ape or preferably a human, most preferably an adult.

Further, the full thickness skin sample may be a punch biopsy. In this context, a “punch biopsy” refers to a skin sample created with a tool important in medical diagnostics—also called biopsy puncher—which is able to punch out/stamp out pieces of skin with cleanly defined diameter. Preferably, the punch biopsy created with a biopsy puncher comprises all skin layers as mentioned above and is therefore a representative cross section of the entire skin (such as epidermis, dermis, subcutis, fascia). A standard biopsy puncher makes it possible to perform skin biopsies in various locations with great precision and minimal tissue trauma. Thus, a punch biopsy as used in the present invention may replace extensive animal use with miniscule tissue biopsies that generate authentic scars.

Preferably, a disposable, round biopsy puncher with 2 mm in diameter may be used. It generates uniform round shape full thickness skin biopsies (punch biopsies) that reduce variability of the method generating an ex vivo skin sample being capable of developing scar. With a custom made puncher it should be possible to go down to 1 mm, yet below that size technical difficulties might occur with sectioning. Additionally, it may also be possible to cut the skin tissue with surgical scissors/scalpels to create preferred full thickness skin samples. These samples will also generate scars, but greater variability may be expected.

Preferably, the punch biopsy is from a dorsal region. Thus, back skin may be collected from C57BL/6J or En1^{Cre}:R26^{mTmG} or En1^{Cre}:R26^{VT2/GK3} mice at different ages, before they are washed twice with cold DMEM/F12 to remove contaminating blood and before round-full thickness skin pieces are created with a biopsy puncher, preferably 2 mm in diameter.

By collecting the back skin is meant taking the entire back skin from the postnatal mice (P0-P2). Thereby, the front border of the back skin is the shoulder (position at the body-end of forelimbs), the rear border border is after the position at the body-end of hindlimbs (before the tail). The left-and-right border is the middle line of dorsal-ventral axis, where the dorsal region starts pigmentation.

For middle dorsal skin region best scaring may be observed. Other regions such as scalp or oral cavity may also be preferred, whereas for scalp region a better scaring is observed compared to oral cavity being used.

Additionally, the full thickness skin sample may be a fresh sample. In this context, a “fresh sample” refers to a skin sample, which is used directly after the skin tissue has been collected from e.g. mice for the method as mentioned above and for the preparation comprising a full thickness skin model comprising a full thickness skin sample. The fresh sample may be free of any contamination.

Preferably, the mammal, where the full thickness skin sample is obtained from may be a mouse or human.

Preferably, C57BL/6J mouse strain may be used as well as En1^{Cre};R26^{mTmG} mouse strain, whereby En1^{Cre} transgenic mice were crossed with ROSA26^{mTmG} reporter mice or En1^{Cre};R26^{Vt2/GK3} mouse strain, whereby En1^{Cre} transgenic mice were crossed with ROSA26^{VT2/GK3} reporter mice.

Human skin tissue may be derived from human fetal tissue (aborted fetus) or adult human tissue, particularly after plastic surgeries, e.g. body lift operations after liposuction. Preferably, human skin tissue may be derived from adult human tissue (liposuction). Preferably, from an adult of 35 to 55 years, more preferably from an adult of 40 to 50 years, most preferably from an adult of 40 years. Adult human tissue may include skin from the back, arm, thigh, and breast regions (in particular breast reduction surgery)—all tested regions provide scar formation which is sufficient for validation experiments with compounds obtained by the mouse assay (although not as distinctive as in case of pre-/neonatal mice).

Tissues from human keloid scars, human hypertrophic scars or any other human dermatologic syndrome may be used to generate SCAD and to study and develop targets against any unique pathobiology features of these dermatologic conditions. By obtaining a skin tissue from adult human tissue, a full thickness skin sample may also be created. In human fetal tissue (aborted fetus of about 10 weeks) no scar development may occur, since epidermis is not yet fully developed at this age, thus fibroblasts tend to adhere to the plastic culture dish, which destroys the 3D properties of fibroblasts.

The mouse may be in a developmental fetal stage of at least E18.5 up to neonatal stage P10. According to Theiler 1989, “The House Mouse, Atlas of embryonic development”, Springer-Verlag, New York mouse development is divided into 26 fetal (prenatal) and 10 neonatal (postnatal) stages. The 26 fetal stages comprises inter alia Theiler stage 1 and 2, which refer to developmental fetal day 1 (E1), comprising the fertilization (Theiler stage 1) and the development of a one-celled egg (Theiler stage 2; first cleavage of the egg occurs at about 24 hours after fertilization) up to Theiler stage 26 (E18, 18 days after fertilization/post coitum), where the whiskers, which have already been visible as short filaments at 17 days, are now longer, the skin is thickened and eyes are barely visible.

The neonatal stage refers inter alia to stage 28 (or P1), the newborn mouse. The neonatal stages P2, P3, P4, P5, P6, P7, P8, P9, and P10 comprise the postnatal development of the mouse in details.

“Fetal” refers to relating to, or having the character of a fetus. Fetal stage in mouse development may also refer to a prenatal stage, wherein “prenatal” refers to occurring, existing, or used before birth. “Neonatal” in this context refers to or relates to a newborn mouse. Neonatal stage in mouse development may also refer to a postnatal stage, wherein “postnatal” refers to occurring, existing, or used after birth. Preferably, the mouse is in a developmental fetal stage of at least E18.5, E19, E19.5, E20, E20.5, E21, E21.5, E22, E22.5, E23, E23.5, E24, E24.5, E25, E25.5 or E26, or in a developmental neonatal stage of at least P1, P1.5, P2, P2.5, P3, P3.5, P4, P4.5, P5, P5.5, P6, P6.5, P7, P7.5, P8, P8.5, P9, P9.5 or P10. The mouse may be in a developmental fetal stage of at least E18.5 up to neonatal stage P9, or in a developmental fetal stage of at least E18.5 up to neonatal stage P7 or in a developmental fetal stage of at least E18.5 up to neonatal stage P5. Preferably, the mouse may be in a developmental fetal stage of at least E18.5 up to neonatal stage P2.

The developmental fetal stage 18.5 (E18.5) refers to 18.5 days after fertilization (post coitum). Normally, E-date is given in day x.5, because mice are active during the night and sleep during the day, assuming fertilization starts in the middle of the night. Since experiments may then start during the day, a half day should be considered when referring to E dates. At the developmental fetal stage E18.5 elongating duct has now grown into the fat pad and has branched into a small ductal system. Cells of the mammary mesenchyme have formed the nipple, which is made of specialized epidermal cells.

At earlier ages (<E18.5) there may not be scaring but regeneration happening, and at later ages (>P2), developed scar tissue using the method of the present invention may be too small to provide a suitable therapeutic window to assess the effects of tested compounds mentioned elsewhere herein. Thus, less scar formation is observed at the ventral side of the skin biopsy with the method of the present invention, if dorsal skin from adult mice (8 weeks), which are reproductively mature is used.

Scar tissue is rarely observed in lower vertebrates where the normal response to injury is a complete regeneration of the original dermal structure. Scarring is however frequent in mammals which have evolved to heal with scar tissue (Gurtner et al. 2008, Nature 453, 314-321 and Ud-Din et al. 2014, Exp. Dermatol. 23, 615-619). Mammals undergo a regeneration-to-scar phenotypic transition during fetal life. This transition has been documented in the back-skin of all mammalian embryos studied to date, including mice, rats, marsupials, rabbits, pigs, non-human primates, and in human fetuses that had undergone corrective spinal surgery for Spina bifida. In the first two trimesters of fetal life (gestational stage E16.5 in mice), injuries regenerate without scarring, as the wounded dermis regenerates the ‘basket-weave’ architecture of intact dermis. From the third trimester (gestational stage E18.5 in mice) on and throughout adulthood, humans and most mammals patch wounds with scars, which are tightly packed parallel collagen bundles and very unlike the original reticular structure.

According to the inventors of the present invention, it was found that from skin sample of earlier developmental stages such as E15.5, E16, E16.5 scars did not develop using the method of the present invention.

Additionally, the full thickness skin sample of the present invention may have an average thickness of about 1 to 3 mm. The full thickness skin sample of the present invention has an average thickness of about 1 mm, 2 mm or about 3 mm. Preferably, the full thickness skin sample of the present invention has an average thickness of about 2 mm. In this context, the term “average thickness” may also refer to “average diameter”.

Further, the present invention comprises liquid culture, wherein the liquid culture may be suspension culture. If a skin sample or cells in general is/are cultured free-floating in the liquid culture (culture medium) it refers to a suspension culture, whereas if growing cells culture as monolayers on an artificial substrate it refers to adherent culture. The skin sample (punch biopsy) may be cultured individually in each well of a multi-well plates, such as 96-well plates (with about 200 μl medium per well), or 384-well plate (with about 80 μl medium per well), preferably it is cultured in 96-well plates. Thus, each well of the 96-well plate or the 384-well plate comprises at least one skin sample. With a single back skin tissue 30 to 300 SCAD experiments (skin sample/punch biopsy developing a scar), preferably 40 to 200 SCAD experiments, more preferably 50 to 100 SCAD experiments are generated, thereby significantly reducing the number of animals to be used to a bare minimum. For large scale culture, multiple skin samples may be cultured in a single culture dish (eg. 10 cm dish) or a bag.

Additionally, the full thickness skin sample may be cultured for at least 4 days. The full thickness skin sample may be cultured for at least 5 days, for at least 6 days, for at least 7 days, for at least 8 days, for at least 9 days, for at least 10 days. Preferably, the full thickness skin sample is cultured for 4 to 10 days. More preferably, the full thickness skin sample is cultured for 5 to 8 days. During culture, old medium is removed and fresh medium is provided with a multi-channel pipette.

By culturing murine full thickness skin sample, fresh medium may be provided every second day and the murine full thickness skin sample may be cultured for at least 4 days, for at least 5 days, for at least 6 days. Preferably, the murine full thickness skin sample is cultured for 4-6 days, more preferably the murine full thickness skin sample is cultured for 5 days. From day 7 degradation of murine full thickness skin sample starts.

By culturing human (adult) full thickness skin sample, fresh medium may be provided every day since human adult skin tissue is much thicker than mouse tissue and consumes nutrients faster. The human full thickness skin sample may be cultured for at least 4 days, for at least 5 days, for at least 6 days, for at least 7 days, for at least 8 days, for at least 9 days, for at least 10 days. Preferably, the human full thickness skin sample is cultured for 7-10 days. From day 10 degradation of human (adult) full thickness skin sample starts.

The present invention further comprises that culturing may be performed by using DMEM/F12 medium comprising 10% FBS, 1× penicillin/streptomycin, 1× GlutaMAX and 1× MEM non-essential amino acids.

Serum-free DMEM/F12 medium may also be used for culturing. In this context, the term “serum-free medium” may refer to a medium (particularly DMEM/F12 medium) in the absence of serum, yet containing a supplement containing defined concentration of growth factors such as GlutaMAX (preferably 1× GlutaMAX), and MEM non-essential amino acids (preferably 1× MEM non-essential amino acids) and some antibiotics (preferably 1× penicillin/streptomycin), thus culturing with DMEM/F12 medium comprising 1× GlutaMAX, and 1× MEM non-essential amino acids and 1× penicillin/streptomycin. In this context, the term “non-essential amino acids” refers to naturally occurring amino acids, that the human body can synthesize for itself, and so need not be provided by dietary protein, such as alanine (Ala), asparagine (Asn), aspartic acid (Asp), cysteine (Cys), glutamic acid (Glu), glutamine (GIn), glycine (Gly), proline (Pro), serine (Ser), tyrosine (Tyr). Using serum-free medium, preferably serum-free DMEM/F12 medium, may show less scar formation though in comparison to medium (DMEM/F12 medium) supplemented with 10% FBS.

In contrast, using basic DMEM/F12 medium only for culturing may not give satisfactory results in terms of scar formation in comparison to medium (DMEM/F12 medium) supplemented with 10% FBS, but still the system works in basic media too. “Basic” DMEM/F12 medium may refer to DMEM/F12 medium in the absence of any supplement.

Further, culturing may be performed in a normal 37° C., 5% CO₂ incubator. Culturing under hypoxic conditions (3.5% O₂), normoxic conditions or even hyperoxic conditions (95% O₂) may also be performed. All tested conditions may result in scar development. Preferably, culturing may be performed in a normal 37° C., 5% CO₂ incubator, in which O₂ is the same as air (−20%).

The present invention also comprises a method for generating an ex vivo skin sample being capable of developing scar, comprising determining whether the full thickness skin sample may contain cells expressing CK14, Engrailed-1 (En1), CD26, N-Cadherin, alpha-smooth muscle actin (α-SMA), fibroblast specific proteins 1 (FSP1) and/or platelet derived growth factor receptors alpha (PDGFRα) and beta (PDGFRP). In this context, the term “expressing” refers to cells “expressing” a surface or cytoplasmic marker such as CK14, CD26, N-Cadherin, α-SMA, FSP1, PDGFRα, PDGFR@ or said term refers to cells “having expressed” when referring to a lineage marker such as En1. Thus, the scar is produced by EPFs (having expressed En1 in the history) expressing CD26, N-Cadherin, α-SMA, FSP-1 or PDGFRα, PDGFRR. CK14 kerationcytes are not responsible directly to produce scar, but it is believed that keratinocytes need to be present in the system to produce the scar that is observed. FIG. 6 shows that at day 5, the keratinocytes migrate over the ventral side of biopsy, mimicking the “re-epithelialization” process of natural wound healing. The fibroblasts presented at the newly formed scar center express CD26, α-SMA and N-Cadherin, which can be used as markers to evaluate the scar formation.

The Engrailed-1-lineage-positive fibroblasts or Engailed1-history-past fibroblasts (EPFs) are the main contributor of scar tissue development in murine back skin and cranial dermis, whereas Wnt1 lineage positive fibroblasts are the main contributor of scar tissue development in murine oral cavity. This embryonic lineage within the dorsal dermis possesses many of the functional attributes and characteristics such as the similar spindle-shaped morphology commonly associated with the term “fibroblast”. However, this lineage is not only present in the skin but also in the underlying superficial fascia. These fibroblast lineages (e.g., EPFs) responsible for scar deposition are derived from circulating fibroblast-like cells. EPFs may refer to En1-lineage-positive fibroblasts, meaning the ancestor/progenitors expressed En1 in the history during embryogenesis, but EPFs most likely do not express Engrailed-1 (En1) at stage of E18.5-P10, the developmental stages where the skin tissues may be collected from mice.

Engrailed-1 (and Wnt1) is expressed only transiently during embryonic development. En1 is a transcription factor, it turns on very early during development and regulates the expression of several downstream target genes. The En1 gene marks a lineage of cells. Once it is turned on, the cells and its progeny are EPFs, no matter whether En1 is expressed or not in the cells. Therefore, En1 is not a surface marker to mark the cells, but a lineage marker, thus defining an embryonic lineage.

With the definition of embryonic lineage, it is not possible to purify EPFs from a wild type mouse or from a human by using En1 as a surface marker. To achieved this, transgenic mouse lines (lineage tracing reporter mouse line) such as En1^{Cre};R26^{mTmG} may be used that traces the embryonic progenitors expressing En1 with a fluorescent reporter (GFP) and which migrate during embryonic development from the somites into the dorsal trunk dermis. The purification is based on the changes of fluorescent reporter from red fluorescent protein (RFP) to green fluorescent protein (GFP). The cells that have never expressed En1 in the history are red. Those cells refer to En1-lineage-naive fibroblasts (in this case ENFs being RFP labeled). The colour change happens at the En1 expression, which is a single event in history. After a short period the En1 expression shuts down, but in this mouse reporter system, all the progeny cells from the ancestor that turned on En1 in the past are permanently green (in this case EPFs being GFP labeled). This refers to the lineage tracing technique, which clearly allows visualizing the progeny of these En1 progenitors in the back skin of mice. These progeny cells are the scar producers, but they no longer express En1. So GFP only marks the event of turn on of En1 in the history, but not a direct label of En1 expression in the cell.

In the wild type mouse system or in human, there is no direct way to mark EPFs. Therefore, surrogate markers such as CD26 or other fibroblast markers as mentioned below may be used for marking EPFs. CD26 labels a large percentage of EPFs (94%) and offers the highest-fold enrichment of EPFs over ENFs that have never expressed Engrailed in the history. ENFs do not participate in scar tissue formation. By transplanting adult ENFs & EPFs, separately, in different anatomical locations, it has been determined that the difference in the capacity of EPFs & ENFs to form a scar is cell-intrinsic, and permanent, and that these are in vivo behaviors of two distinct fibroblastic cell types (Rinkevich et al., 2015, Science 348 (6232)).

For human samples, the bellow pan markers for fibroblasts may further be used, such as N-Cadherin, alpha-smooth muscle actin (α-SMA), fibroblast specific protein 1 (FSP1), and/or platelet derived growth factor receptors alpha (PDGFRα) and beta (PDGFRβ), all important indicators and markers of scar formation. The bellow pan markers for fibroblasts as mentioned above may also be used in mice as well.

Determining whether the full thickness skin sample contains cells expressing Engrailed-1, CD26, N-Cadherin, alpha-smooth muscle actin (α-SMA), fibroblast specific protein 1 (FSP1), or platelet derived growth factor receptors alpha (PDGFRα) and beta (PDGFRβ) or even keratinocytes expressing CK14 may provide a quality control measure which allows a skilled person to check the quality/potential of the skin sample to develop scars.

Preferably, said full thickness skin sample of the present invention may be characterized by cells expressing α-SMA, CD90, ER-TR7, PDGFRα, Sca1, βIIITubulin, CD31, MOMA-2, F4/80, CD24, CD34, CD26, Dlk, Fn1, Col14a1, Emilin2, Gsn and/or Nov (FIGS. 9, 10, 11 and 12).

Additionally, the present invention comprises the method for generating an ex vivo skin sample being capable of developing scar, wherein in step (b) determining whether a scar is developed by the full thickness skin sample in step (a) may be done by visual inspection.

The term “visual inspection” refers to the visualization of the full thickness skin sample by for example using a stereomicroscope (such as Leica M50), Zeiss AxioImager microscope or laser scanning microscope thereby determining whether said sample (punch biopsy) may have developed a scar. The typical scar develops at the center of the ventral side of the biopsy, has opaque whitish soft tissue morphology, with hair follicles at the boundary. With time, fibril cytoarchitectures increase in complexity, and expanding sub-ventrally a matrix that bridged between lateral sides of the biopsy. The percentage of developed scar after visualizing the full thickness skin sample may be derived from the whole-mount pictures taken on day 5 after culturing, since the border of the scar, and the border of the tissue can be seen from the images, and the percentage can be derived from those areas.

1-10 μm, 2-8 μm, or 4-6 μm, or 1, 2, 3, 4, 5, 6, 7, 8, 9, or 10 μm cryosections generated from the paraformaldehyde fixed punch biopsy already being cultured before fixation as mentioned elsewhere herein may be stained with Masson's trichrome staining, resulting in blue color stained collagen bundles at the scar region making it easier for visualization of scars using a stereomicroscope. Preferably, 6 μm cryosections may be used for staining. Masson's trichrome staining is a three-colour staining protocol used in histology, which selectively stain muscle, collagen fibers, fibrin, and erythrocytes. Bouin's solution is used first as a mordant to link the dye to the targeted tissue components. Nuclei are stained with Weigert's hematoxylin, an iron hematoxylin, which is resistant to decolorization by the subsequent acidic staining solutions. Biebrich scarlet-acid fuchsin solution as the frist colour of masson's trichrome staining stains all acidophilic tissue elements such as cytoplasm, muscle, and collagen. Subsequent application of phosphomolybdic/phosphotungstic acid is used as a decolorizer causing the Biebrich scarlet-acid fuchsin to diffuse out of the collagen fibers while leaving the muscle cells red. Application of aniline blue as the third colour of masson's trichrome staining will stain the collagen, after which, 1% acetic acid is applied to differentiate the tissue sections. Thus, masson's trichrome staining produces red keratin and muscle fibers, blue or green collagen and bone, light red or pink cytoplasm, and dark brown to black cell nuclei.

Since dermal structure is too complex to be analyzed with simple Euclidean geometry parameters such as length, area, and angles, dermal lattice development fractal analysis (also called complexity analysis) to measure the complexity of arrangements of cells and of matrix fibers may also be used in the present invention for visual inspection as well (using adapted script called plug-in “FracLac V 2.0f” for Image J software). Fractal dimensions (FD) and lacunarity (L) are two values derived from the fractal analysis that have been used to assess and quantify morphologically complex objects like vessels and tumors (Gould et al. 2011, Microcirculation. 18, 136-151).

Complex cellular arrangements, such as blood vessels, score higher FD values, while simpler arrangements (e.g. geometrical shapes) score lower FD values.

On the other hand, L values reflect the “gappiness” or empty spaces between shapes (porosity). Porous structures (eg. sponges) score higher L values than smooth surfaces (eg. scales). Thus, these two complementary values, together, may be used to describe the general organization of dermal structures from two-dimensional images. By using the method of fractal analysis on histological slides generated from SCAD, the extent and severity of scar tissue development may be accurately determined.

Further, the present invention envisages a method for generating an ex vivo skin sample being capable of developing scar, wherein in step (b) determining whether a scar is developed by the full thickness skin sample in step (a) comprises determining whether collagen type I, collagen type III and/or fibronectin may be present in said full thickness skin sample.

Besides visual inspection using Masson's trichrome staining or fractal analysis, immunolabeling of the major fibril proteins of extracellular matrix (ECM) may also be applied in the present invention.

Using antibodies targeting collagen type I, collagen type III and fibronectin, which are extracellular matrix (ECM) fiber proteins abundantly presented in the scar tissue may be used for immunolabeling method (FIG. 5).

Visualization/confirmation may therefore be performed with histological staining for ECM proteins (eg. Masson's trichrome staining for collagens), or immunofluorescence staining of these ECM fiber proteins such as collagen I and III and fibronectin.

SCAD's extracellular fiber alignment and architecture matches those that develop during in vivo scars. SCAD therefore epitomizes in vivo scars in composition, structure and cellular origin.

The present invention further comprises a method for screening for a compound which may modulate scar development, comprising a) carrying out the method of the present invention in the presence of a compound of interest; and b) determining whether said compound of interest modulates scar development in comparison to carrying out the method of the present invention in the absence of said compound of interest.

The term “screening” refers to testing objects such as compounds of interest in order to identify those with particular characteristics (e.g, being able to modulare scar development).

The term “compound” may refer to an inhibitor, thus inhibiting scar development of the punch biopsy, once the inhibitor is applied to the sample. The term “compound” may also refer to a promoter/an inducer, thus promoting/inducing scar development of the punch biopsy, once the promoter is applied to the sample.

The term “modulate” as used herein means “inhibit”, if the compound may be an inhibitor of scar development or “promote/induce”, if the compound may be a promoter/an inducer of scar development.

Modulation of scar development may therefore be inhibition of scar development or promotion of scar development.

Modulation of scar development was performed using Nefopam hydrochloride (Prestw-229), the positive control known to reduce scar formation, thus being an inhibitor of scar development (FIG. 7). Nefopam, sold under the brand name Acupan, among others, is a pain killing medication used to treat moderate, acute or chronic pain. It is believed to work in the brain and spinal cord to relieve pain. Firstly it increases the activity of the serotonin, norepinephrine and dopamine, neurotransmitters involved in, among other things, pain signaling. Secondly, it modulates sodium and calcium channels, thereby inhibiting the release of glutamate, a key neurotransmitter involved in pain processing.

Said method may comprise administering the compound to a subject. The subject may be a mammal, preferably a mouse. The administration of a compound mentioned above may be performed by injection or by infusion. Preferably, said administration of a compound mentioned above may be performed intradermally (by intradermal injection).

### Preparation

Additionally, the present invention further encompasses a preparation comprising a scarred full thickness skin sample obtainable by the method of the present invention.

The term “preparation” may be used interchangeably with the term “composition”.

The term “scarred full thickness skin sample” refers to the skin (sample) comprising all skin layers as mentioned elsewhere herein and being cultured immersed and untethered in liquid culture having then developed a scar.

It is envisaged that the preparation of the present invention may comprise the scarred full thickness skin sample and a carrier in any combination. The preparation comprising the scarred full thickness skin of the present invention, if desired, may also comprise amounts of wetting or emulsifying agents, or pH buffering agents as a carrier. Preferably, the carrier is PBS. The preparation may be in solid or liquid form or even frozen. Also it is encompassed that the preparation comprises the scarred full thickness skin being available as 1, 2, 3, 4, 5, 6, 7, 8, 9, or 10 μm cryosection, or as 1-10 μm, 2-8 μm, or 4-6 μm cryosection, preferably as 6 μm cryosection.

Additionally, the scarred full thickness skin sample of the preparation may be stained with masson's trichrome staining.

Thus, the preparation comprising the scarred full thickness skin of the present invention is, for example, suitable for use in immunoassays in which it may be utilized in liquid phase or bound to a solid phase carrier. Examples of well-known carriers include glass, polystyrene, polyvinyl ion, polypropylene, polyethylene, polycarbonate, dextran, nylon, amyloses, natural and modified celluloses, polyacrylamides, agaroses, and magnetite. The nature of the carrier can be either soluble or insoluble for the purposes of the invention. Solid phase carriers are known to those in the art and may comprise polystyrene beads, latex beads, magnetic beads, colloid metal particles, glass and/or silicon chips and surfaces, nitrocellulose strips, membranes, sheets, duracytes and the walls of wells of a reaction tray, plastic tubes or other test tubes. Suitable methods of immobilizing the scarred full thickness skin of the present invention on solid phases include but are not limited to ionic, hydrophobic, covalent interactions or (chemical) crosslinking and the like. Examples of immunoassays which can utilize the scarred full thickness skin of the present invention are competitive and non-competitive immunoassays in either a direct or indirect format. Commonly used detection assays may comprise radioisotopic or non-radioisotopic methods. Examples of such immunoassays are the radioimmunoassay (RIA), the sandwich (immunometric assay) and the Northern or Southern blot assay. Furthermore, these detection methods comprise, inter alia, IRMA (Immune Radioimmunometric Assay), EIA (Enzyme Immuno Assay), ELISA (Enzyme Linked Immuno Assay), FIA (Fluorescent Immuno Assay), and CLIA (Chemioluminescent Immune Assay).

Thus, the present invention may comprise the preparation comprising a scarred full thickness skin sample obtainable by the method for generating an ex vivo skin sample being capable of developing scar, further comprising wetting or emulsifying agents, or pH buffering agents.

The present invention further comprises a preparation comprising a full thickness skin model comprising a full thickness skin sample immersed and untethered in liquid culture.

In this context, “a full thickness skin model” refers to the formulation of a full thickness skin sample as described elsewhere herein, where preferably non-skin tissue may have been carefully removed from (e.g., with a surgical scalpel) and which preferably has an average diameter of 1 to 3 mm (preferably 2 mm), and liquid culture, where the full thickness skin sample is cultured immersed and untethered in.

Preferably, liquid culture is suspension culture. More preferably, DMEM/F12 medium comprising 10% FBS, 1× GlutaMAX, 1× penicillin/streptomycin, and 1× MEM non-essential amino acids is used for suspension culture in the preparation comprising a full thickness skin model.

The preparation comprising a full thickness skin model may be in liquid form available in wells (96-wells or 384-wells), in single culture dishes (eg. 10 cm dish) or even in bags.

The present invention further comprises the preparation comprising a full thickness skin model comprising a full thickness skin sample for use in a method for screening for a compound which modulates scar development.

Envisaged by the present invention may also be the preparation comprising a full thickness skin model comprising a full thickness skin sample for use in therapy or diagnosis.

Preferably, the preparation comprising a full thickness skin model comprising a full thickness skin sample is used in therapy or diagnosis of any one of hypertrohic scar, kloid scar, large burns, chronic wounds, systemic sclerosis, scleroderm, acne, stretch marks after weight gain or pregnancy, or chickenpox.

The present invention also comprises a preparation comprising (a) compound(s) modulating scar development. Preferably, a preparation comprising (a) compound(s) inhibiting scar development. In this context, the term “preparation” may refer to an ointment formulation. The present invention envisages that the preparation mentioned above is administered to a subject. The subject may be any subject as defined herein. Preferably, the subject is a mammal, more preferably the subject is a human, most preferably the subject is an adult. The subject is preferably in need of the administration. The present invention encompasses that the application is performed by topical application. In this context, the term “topical application” refers to applied to, or affecting a localized area of the body, especially of the skin. And by affecting a localized area of the body, the applied substance (agent) may only act at this specific location during topical application. Thus, the risk of possibly undesired side effects may be reduced by topical application.

The present invention further comprises a method of modulating scar development comprising administering an effective amount of a preparation comprising (a) compound(s) being able to modulate scar development to a subject in need thereof. The subject may be any subject as defined herein.

Further, the present invention may envisage the use of a preparation comprising (a) compound(s) modulating scar development for the manufacture of a medicament for therapeutic application for any one of surgical scar, hypertrohic scar, kloid scar, large burns, chronic wounds, systemic sclerosis, scleroderm, acne, stretch marks after weight gain or pregnancy, or chickenpox.

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention. In the context of the present invention, “at least one” means one, two, three, four, five, six, seven, eight, nine, ten or more.

The term “and/or” wherever used herein includes the meaning of “and”, “or” and “all or any other combination of the elements connected by said term”.

The term “less than” or in turn “more than” does not include the concrete number.

The term “generating” may be used interchangeably with the term “producing” or “creating”.

The term “ex vivo” refers to “in vitro” and may be used interchangeably.

For example, less than 20 means less than the number indicated. Similarly, more than or greater than means more than or greater than the indicated number.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”. When used herein “consisting of” excludes any element, step, or ingredient not specified.

The term “including” means “including but not limited to”. “Including” and “including but not limited to” are used interchangeably.

The term “about” means plus or minus 10%, preferably plus or minus 5%, more preferably plur or minus 2%, most preferably plus or minus 1%.

Throughout the description and claims of this specification, the singular encompasses the plural unless the context otherwise requires. In particular, where the indefinite article is used, the specification is to be understood as contemplating plurality as well as singularity, unless the context requires otherwise.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications cited throughout the text of this specification (including all patents, patent application, scientific publications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

A better understanding of the present invention and of its advantages will be gained from the following examples, offered for illustrative purposes only. The examples are not intended to limit the scope of the present invention in any way.

### EXAMPLES OF THE INVENTION

### Material and Methods

### Example 1: Mice and Human Samples

Mice were bred and maintained at the Helmholtz Center Munich Animal Facility in accordance with the protocol approved by the local Ethical Committee with the approval number 55.2-1-54-2532-61-2016. All the animals were housed in sterile micro-insulators and given water and rodent chow ad libitum. C57BL/6J, and En1^{Cre} strains were obtained from Jackson laboratories. The ROSA26^{mTmG} (R26^{mTmG}) and ROSA26^{VT2/GK3} (R26^{VT2/GK3}) reporter mice were obtained from Stanford University. En1^{Cre} transgenic mice were crossed with R26^{mTmG} or R26^{VT2/GK3} reporter mice. En1^{Cre};R26^{mTmG} or En1^{Cre};R26^{VT2/GK3} offspring were used to trace En1-lineage positive fibroblasts (EPFs) (Rinkevich et al., 2015).

For experiments using human skin samples, informed consent for the studies was obtained from the patients in accordance with the Declaration of Helsinki and IRB approval of Rechts der Isar Hospital.

### Example 2: Scar-in A-Dish (SCAD) Culture

Dorsal skins were collected from C57BL/6J or En1^{Cre};R26^{mTmG} or En1^{e};R26^{VT2/GK3} mice at different ages as indicated, and washed twice with cold DMEM/F12 (Thermo Fisher Scientific 11320074) medium to remove contaminating blood, and washed once with Hank's Balanced Salt Solution (HBSS, Thermo Fisher Scientific 14175095). After careful removal of non-skin tissue at the ventral side with a surgical scalpel, round full-thickness skin pieces were created with disposable Ø 2 mm biopsy punch (Stiefel 270130), and cultured in 300 μl of DMEM/F12 medium containing 10% FBS, 1× GlutaMAX (Thermo Fisher Scientific 35050038), 1× Penicillin/streptomycin (Thermo Fisher Scientific 15140122), and 1× MEM non-essential amino acids (Thermo Fisher Scientific 11140035) in each well of 96-well plates in a humidified 37° C., 5% CO₂ incubator. (Note: the 2 mm skin pieces were cultured in medium but not floating at the liquid air surface). Fresh medium was supplied every second day and the skin tissues were harvested at the indicated time points (day 1 to day 5 after culture), with the fresh tissues serving as day 0 control, and fixed in 2% PFA for overnight at 4° C.

After washing with PBS, the samples were then embedded and frozen in Tissue-Tek O.C.T (Sakura 4583) in Tissue Tek Cryomold intermediate/normal size depending on the number of samples and 6 μm cryosections were prepared with a cryostat (HYRAX C50). The resulting sections were analyzed by Masson's trichrome staining (Example 3) or immunofluorescent staining (Example 4) and used for data analysis.

### Example 3: Masson's Trichrome Staining

For the Masson's trichrome staining, cryosections were fixed in cold acetone (−20° C.) for 5 min. Air-dry the slides after taking them out of the acetone and then wash with deionized water for 2 mins, before incubating overnight in Bouin's solution (Sigma-Aldrich HT10132) at room temperature. After washing in cold tap water to remove the yellow color from the sections and rinsing in deionized water, the sections were stained with working solution of Weigert's Iron Hematoxylin (Sigma-Aldrich HT1079) for 5 min. Thereafter, the sections were subjected to Masson's trichrome stain kit (Sigma-Aldrich HT15) by sequentially incubating at room temperature in Biebrich Scarlet-Acid Fucshin for 5 min, working solution of Phosphotungstic/Phosphomolybdic acid for 5 min, aniline blue solution for 10 min, and 1% acetic acid for 2 min. Discard the solution. Rinse the slides with distilled water. Dehydrate the slides by placing them sequentially for 5 mins each in 80% EtOH followed by 100% EtOH twice. After dehydration, the sections were cleared with Roti-Histol (Roth 6640) and mounted with Roti-Histokitt (Roth 6638).

### Example 4: Immunofluorescence Staining

Cryosections were fixed in cold acetone for 5 min and blocked with 5% BSA in PBS for 1 h. Thereafter, sections were incubated overnight with primary antibodies at 4° C. Respective isotypes served as negative controls. After washing with PBS for three times, sections were incubated with respective fluorophore conjugated secondary antibodies (Thermo Fisher Scientific) at room temperature for 1 h in a dark chamber. For double staining, sections were incubated at room temperature with the second primary antibody for 2 h, and respective secondary antibody for 1 h in a dark chamber. The dark chamber was filled with Milli Q water to keep the slides hydrated. The slides were then washed with PBS for 5 mins three times and nuclei were counterstained with DAPI for 3 min. Then, coverslips were mounted with Fluoromount-G (Thermo Fisher Scientific 00-4958-02). Photomicrographs were documented by using a Zeiss AxioImager microscope with AxioVision software (Carl Zeiss), or Zeiss laser scanning microscope LSM710 with Zen software (Carl Zeiss) (Example 5).

### Example 5: Microscopy

After fixing the SCAD tissues with 2% PFA overnight (see Example 2), the tissues were washed with PBS for three times and whole mount bright-field images were taken using stereomicroscope Leica M50 at 4× magnification with a Leica DF310 FX camera and using Leice Application Suite (V.4.8).

For capturing images of tissue sections stained with Masson's trichrome dye (Example 3) and for immunohistochemical slides (Example 4) with fine sections, a Zeiss AxioImager microscope with AxioVision software (Carl Zeiss), or Zeiss laser scanning microscope LSM710 with Zen software (Carl Zeiss) was used.

Laser scanning confocal microscopy (also referring to 3D confocal imaging) has become an invaluable tool for a wide range of investigations in the biological and medical sciences for imaging thin optical sections in living and fixed specimens ranging in thickness up to 100 micrometers. The focused beam of the laser scans over the sample and the intensity of the reflected beam is displayed as a function of location to create a digital reflected light image of the sample. This scanning of a focused laser beam thus allows the procurement of digital images with very high resolution as the resolution is finally determined by the position of the beam. Modern LSMs offer a lot of advantages for biological specimens like having the control of the depth of the field, reduction in background fluorescence. Staining was analyzed for skin SCAD sections of 6 μm and 10 μm.

For live imaging of fascia cultures as described in Example 9, samples were embedded as mentioned above. Attention was paid to mount the samples with the fascia facing up towards the objective. Imaging medium (DMEM/F-12; SiR-DNA 1:1,000) was then added. Over-twenty-hour time-lapse imaging was performed under the multi photon microscope. Temperature control was set to 37° C. with 5% CO2-supplemented air. Second harmonic generation and green autofluorescence as a reference were recorded every hour. 3D and 4D data was processed with Imaris 9.1.0 (Bitplane) and ImageJ (1.52i). Contrast and brightness were adjusted for better visibility.

### Example 6: 3D Imaging

Whole mount samples were stained and cleared with a modified 3DISCO protocol. In short, fixed samples were pre-incubated in Dulbecco's Phosphate-Buffered Saline (DPBS, Thermo Fisher Scientific 14190169) containing 0.2% Gelatin (Sigma G1393), 0.5% Triton-X100 (Sigma X100) and 0.01% Thimerosal (Sigma T8784) (PBS-GT). Antibodies anti-elastin (Abcam ab21610), anti-fibronectin (Abcam ab23750) and anti-collagen type I (Rockland 600-401-103) were pre-labeled with Alexa Fluor 488, 594 and 647 dyes (Thermo Fisher Scientific A20181, A20184, A20186) according to the manufacturer's instructions. The samples were incubated with the (labeled) antibodies (f.e. anti-α-SMA, anti-N-Cadherin, anti-elastin, anti-fibronectin, anti-collagen type 1) in PBS-GT (1:1000) in rotation for 24 h at room temperature. After washing in PBS-GT, samples were first dehydrated in an ascending THE (Sigma 186562) series (50%, 70%, 80%, 3×100%; 30 minutes each), than cleared in dichloromethane (Sigma 270997) for 30 min and eventually immersed in benzyl ether (Sigma 108014). Cleared samples were imaged in 35 mm glass bottom dishes (ibidi 81218) using a laser scanning confocal microscope (Zeiss LSM710) (see Example 5), or a multiphoton microscope (Leica TCS SP8). Raw data was processed and optimized for visualization, adjusting brightness and contrast with Imaris 9.1.0 (Bitplane, UK).

For the fascia experiments described in the present invention, the following primary antibodies were used: goat-anti-αSMA (1:50, Abcam), rabbit-anti-ollTubulin (1:100, Abcam), goat-anti-CD29 (1:20, R & D systems), rat-anti-CD90 (1:100, Abcam), rat-anti-CD9 (1:40, Santa Cruz), rat-anti-CD24 (1:50, BD biosciences), rabbit-anti-CD26 (1:150, Abcam), rabbit-anti-CD31 (1:10, Abcam), rat-anti-CD34 (1:100, Abcam), rabbit-anti-Dlk1 (1:200, Abcam), rat-anti-ERTR7 (1:200, Abcam), rat-anti-F4/80 (1:400, Abcam), rabbit-anti-Lyve1 (1:100, Abcam), rat-anti-MOMA2 (1:100, Abcam), goat-anti-Pdgfra (1:50, R & D systems), rat-anti-Sca1 (1:150, Biolegend). PacificBlue-, AlexaFluor488-, or AlexaFluor647-conjugated antibodies (1:500, Life technologies) against suitable species were used as secondary antibodies.

### Example 7: Chemical Screening with the SCAD Assay

D0 SCADs (see Example 2) were first treated with Nefopam from Prestwick chemical library (FDA approved library). The initial concentration of the chemical in library format is 1 mM, dissolved in DMSO. The final concentration of chemical used in FDA-library is 2.5 μM.

All chemicals in the library are the same (1 mM), and they are added by a robot, eg. 0.5 μl chemical per well of 200 μl culture to make 2.5 μM final concentration. SCAD biopsies were prepared in 96-well plate with 200 μl per well, the automated system was used to add the chemical Nefopam.

All the data were compiled on excel sheets and scores were added based on the visual variability observed when compared to the DMSO and media control D5 SCADs.

### Example 8: The Adeno-CMV-eGFP Viral Particles

The adeno associated virus serotype 6 (AAV6) expressing GFP or Cre recombinase being used in Example 20 were produced by transfecting AAVpro® 293T Cell Line (Takara Bio, 632273) with pAAV-U6-sgRNA-CMV-GFP (Addgene, 8545142) or pAAV-CRE Recombinase vector (Takara Bio, 6654), pRC6 and pHelper plasmids procured from AAVpro Helper Free System (Takara Bio, 6651). Transfection was done using PEI transfection reagent and viral harvest was done 72 h post transfection. AAV6 viruses were extracted and purified using AAVpro® purification kit (Takara Bio, 6666) and titre was calculated using real-time PCR.

### Example 9: Fascia In Vitro Cultures

To visualize the changes in the ECM architecture in real time, 2 mm-diameter biopsies were excised from P0 C57BL/6J neonates and processed for live imaging. To determine the effectiveness of the DT treatment, Panniculus carnosus muscle+ superficial fascia was manually separated from the rest of the skin in the chimeric grafts experiments and incubated with DT at different concentrations for 1 h at ambient temperature. Next, samples were washed with PBS and incubated in DMEM/F12 (Thermo Fisher) supplemented with 10% Serum (Thermo Fisher), 1% penicillin/streptavidin (Thermo Fisher), 1% GlutaMAX (Thermo Fisher) and 1% Non-Essential Amino Acids solution (Thermo Fisher) in a 37° C., 5% CO2 incubator. Medium was routinely exchanged every other day. Samples were fixed at day 6 of culture with 2% paraformaldehyde and processed for histology applying the methods as outlined above.

### Example 10: Image Analysis

Histological images were analyzed using ImageJ. For quantification of labeled cells, the wound bed, surrounding dermis and adjacent fascia areas were defined manually. The wound bed was defined as the area flanked by the closest hair follicles on both sides, extending from the base of the epidermis down the dermis to the level were the hair follicles begin. Surrounding dermis was defined as the 200 microns immediately adjacent to the wound bed on both sides, while the fascia was defined as the tissue below the wound bed. The number of labeled cells in each area was determined by quantifying the particles double positive for DAPI in the blue channel and for the label channel. Percentages were presented either as fraction of the total nuclei (DAPI) or fraction of total labeled (DAPI+label) cells in each area.

### Example 11A: Fascia Cells Labelling in Neonates with Adeno-CMV-eGFP

Two 2 mm-diameter full-thickness excisional wounds were created on the back of neonatal C57BL6/J mice (P0) with a biopsy punch (Stiefel, North Carolina). 20 μl of eGFP-expressing adenovirus (Ad-CMV-GFP) at viral titre of 5×10⁹/ml were injected subcutaneously at the area between the two wounds. Wounded tissue was harvested on day 3 and day 7 post-wounding and processed for cryosection to detect the eGFP-expressing cells by fluorescence microscopy.

### Example 11B: Fascia Cells Labelling in Adults with Dil

Two 5 mm-diameter full-thickness excisional wounds were created on the back of 8-10 weeks old C57BL6/J mice with a biopsy punch. 10-20 μl of the lipophilic “Vybrant™ Dil” dye (Life technologies, V22885) were injected into the exposed superficial fascia right bellow the Panniculus carnosus muscles. Wounded tissue was harvested on day 9 and day 14 post-wounding and processed for histology and imaging by fluorescence microscopy.

### Example 12: Chimeric Skin Transplantations Concerning Example 20

Full-thickness 6 mm-diameter biopsies were collected from the back-skin of either R26^{mTmG}, R26^{VT2/GK3}, En1^{Cre};R26^{mTmG}, En1^{Cre};R26^{DTR}, or C57BL6/J adult mice. Using the Panniculus carnosus muscle layer as an anatomical reference, the fascia together with the muscle layer was carefully separated from the dermis and epidermis using Dumont #5 forceps (Fine Science Tools) and a 26 G needle under the fluorescent stereomicroscope (M205 FA, Leica). EPFs from fascia+muscle samples of En1^{Cre};R26^{IDTR} mice were ablated by incubation with 20 μg/ml of diphteria toxin (Sigma-Aldrich, D0564) or only DMEM/F12 as vehicle for 1 h at ambient temperature followed by 3 washing steps with PBS. At this point, muscle+fascia samples-ECM was labeled by incubation with 100 μM Alexa Fluor™ NHS Ester (Life technologies, A20006) in PBS for 1 h at ambient temperature followed by 3 washing steps with PBS. Chimeras were made by placing the epidermis+dermis portion of a mouse strain on top of the muscle+fascia of another strain and left to rest for 20 min at 4° C. inside a 35 mm culture dish with 2 ml of DMEM/F12. Special attention was paid on preserving the original order of the different layers (Top to bottom: Epidermis->Dermis->Muscle->Fascia). Then, a 2 mm “deep” full-thickness was excised from the chimeric graft using a biopsy punch in the middle of the biopsy. To create “superficial” wounds, the 2 mm excision was done only in the epidermis+dermis half, prior to reconstitution with the bottom part. “Wounded” chimeric grafts were then transplanted into freshly-made 5 mm-diameter full-thickness excisional wounds in the back of either RAG2−/− or Fox Chase SCID immunodeficient 8-10 weeks-old mice. Precautions were taken to clean out the host blood from the fresh wound before the transplant and to leave the graft to dry for at least 20 min before ending the anesthesia, to increase the transplantation success. To prevent mice from removing the graft, a transparent dressing (Tegaderm, 3M) was placed on top of the grafts. At dpw 5 and 7, mice received 200 μl i.p. injections of 1 mg/ml EdU in PBS. Samples were collected at dpw 7, 14, and 70 and processed for cryosection and imaging by fluorescence microscopy.

Statistical and bioinformatics operations were run with GraphPad 6 and the Scanpy toolkit.

### Results

### Example 13: Normal Scar Formation in SCAD with Newborn Mice

The first aim was to establish an ex vivo culture method which could be subsequently used later to monitor the effect of chemicals in scarred skin. Newborn wild-type mice (see Example 1, C57BL/6J) back skin was cultured in supplemented DMEM media in 96 well plates for 5 days and the skin samples were embedded in Tissue-Tek O.C.T (see Example 2). The samples were sectioned and stained using Masson's Trichrome staining (see Example 3). Scar development was observed from D0 to D5 by fixing samples at D0, D1, D2, D3, D4 and D5 stages of development.

All the images of tissue sections were taken under 10× magnifications with Axio Imager (see Example 5).

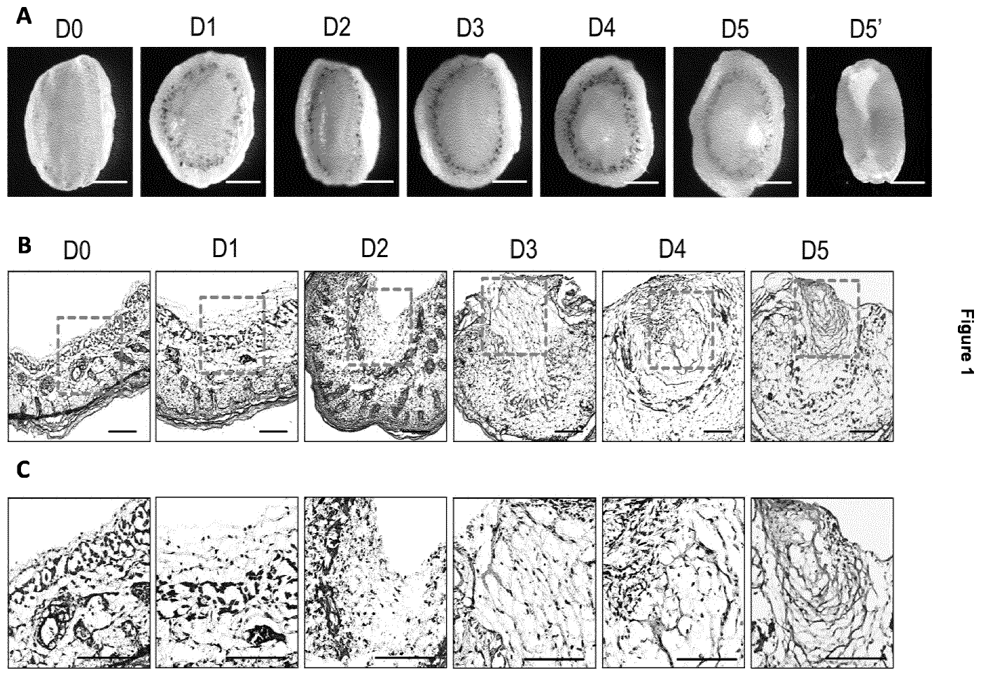

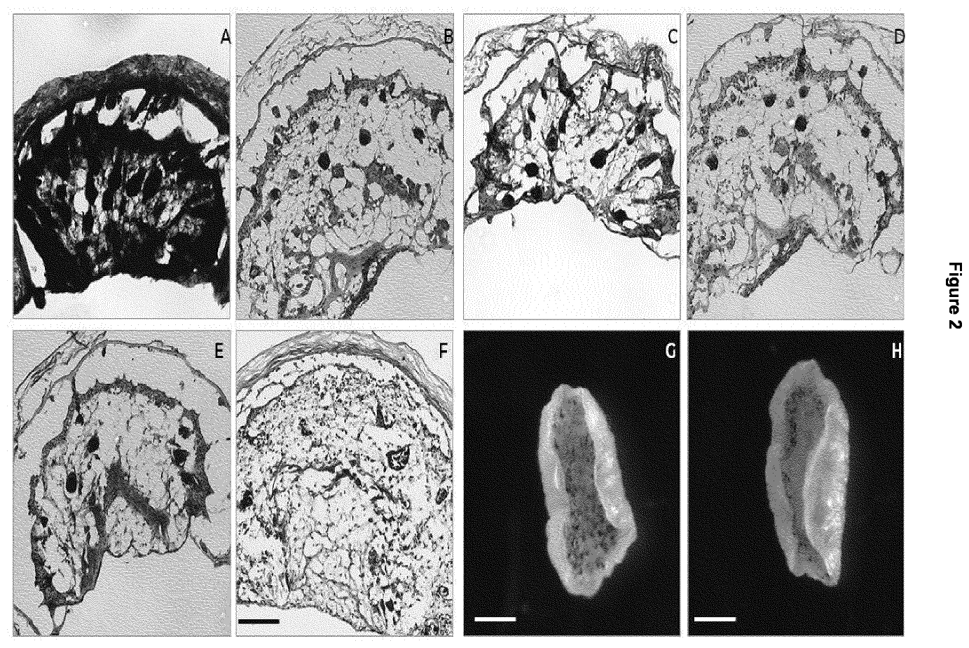

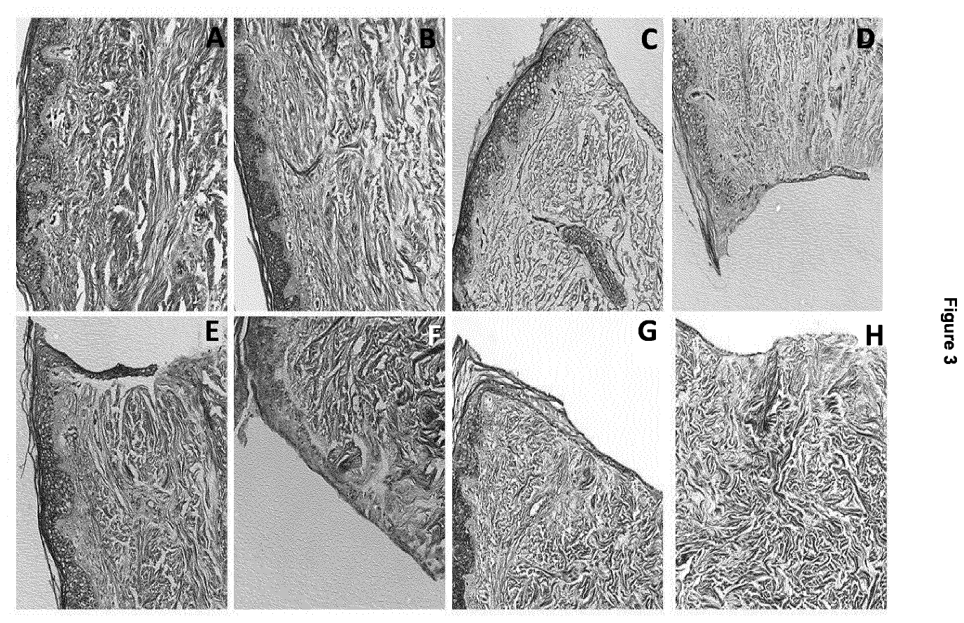

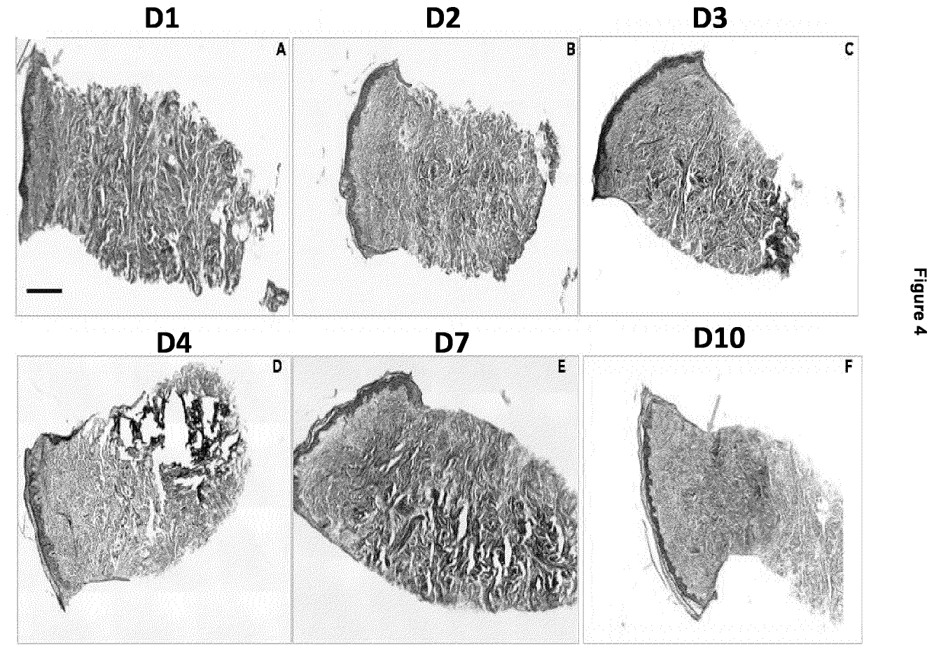

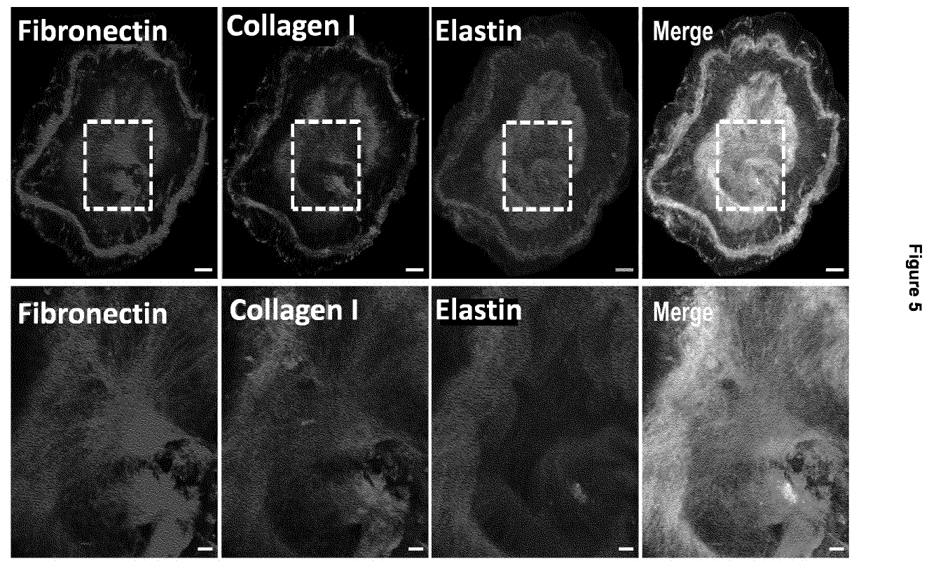

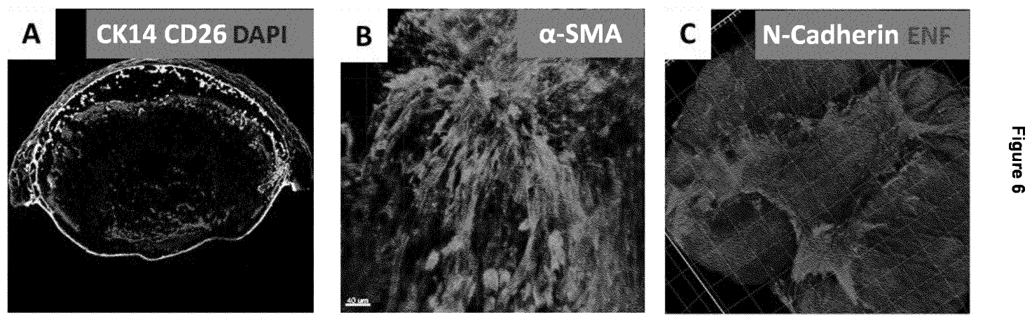

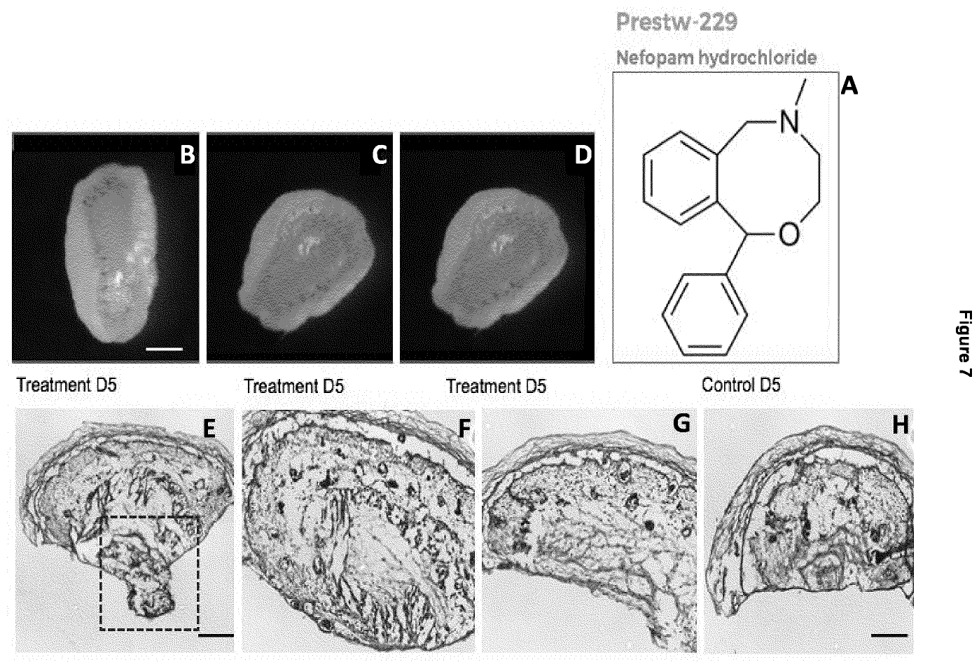

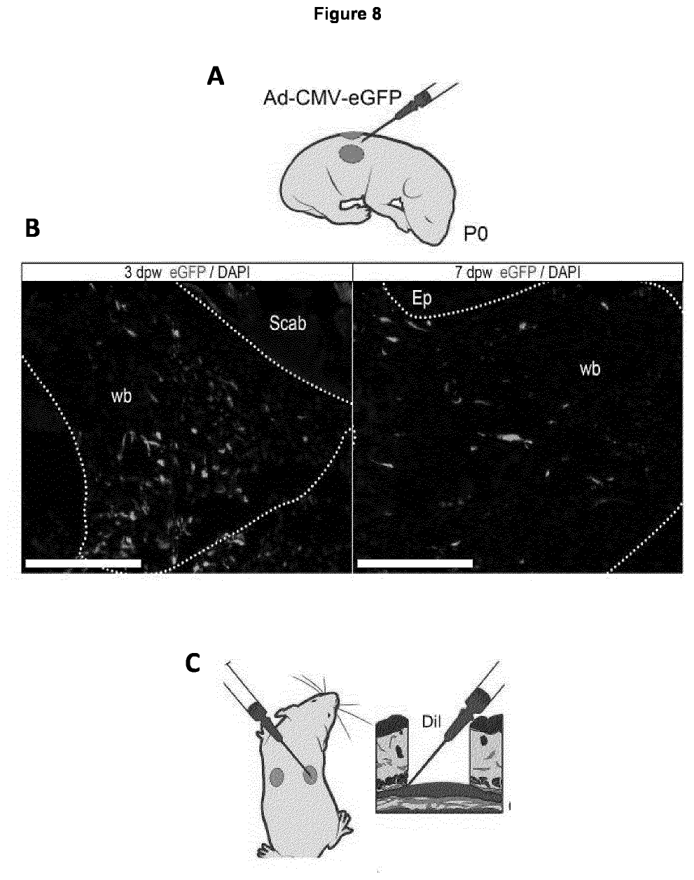

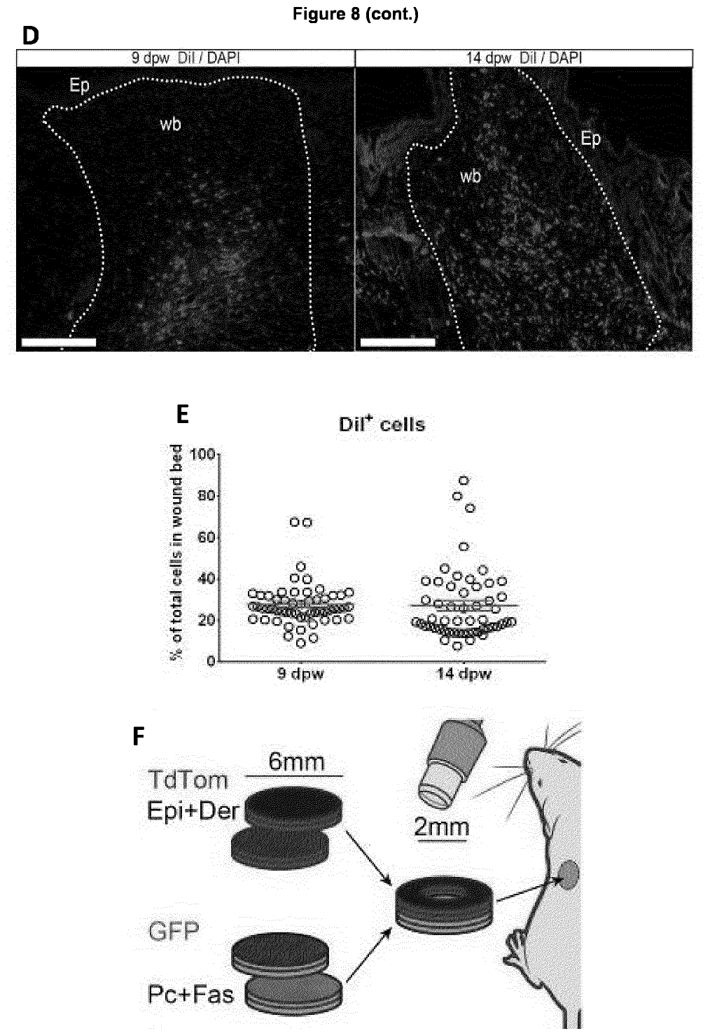

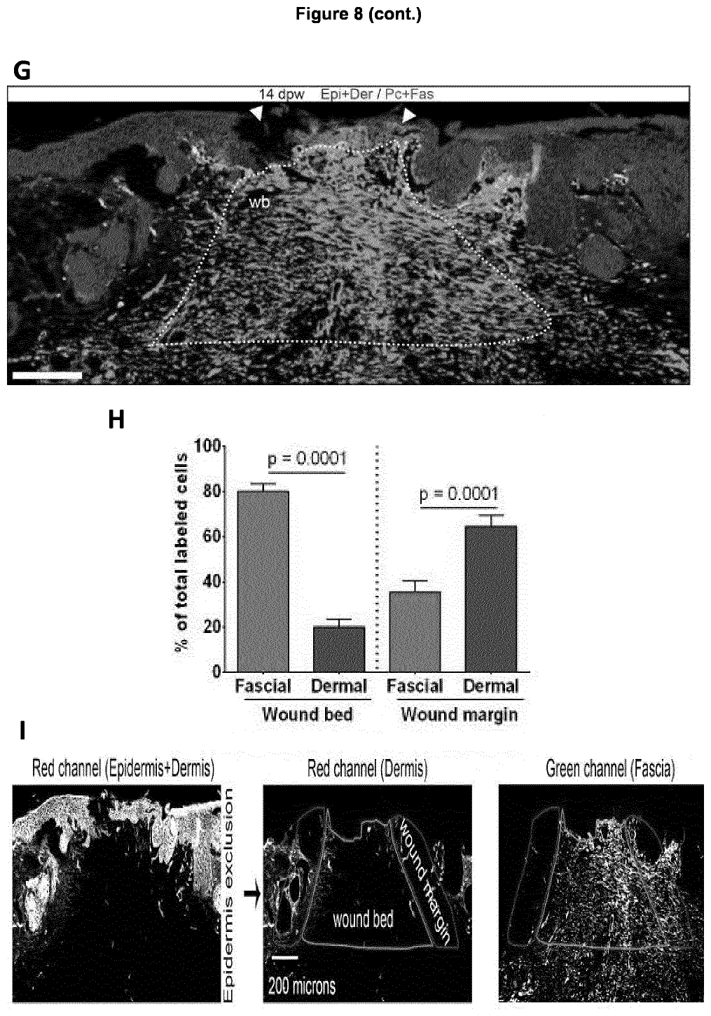

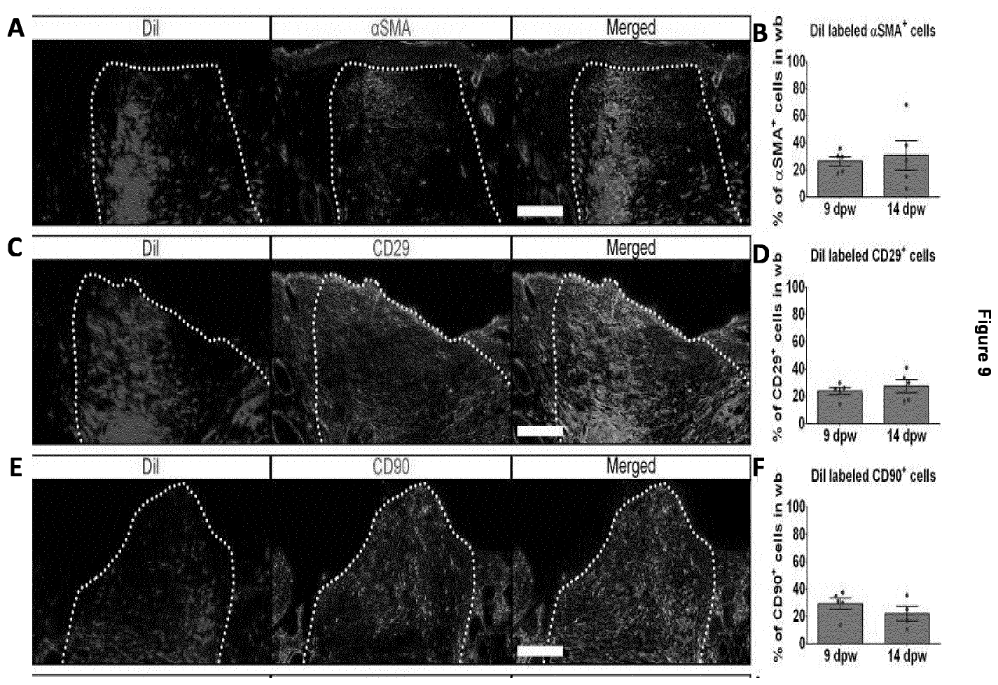

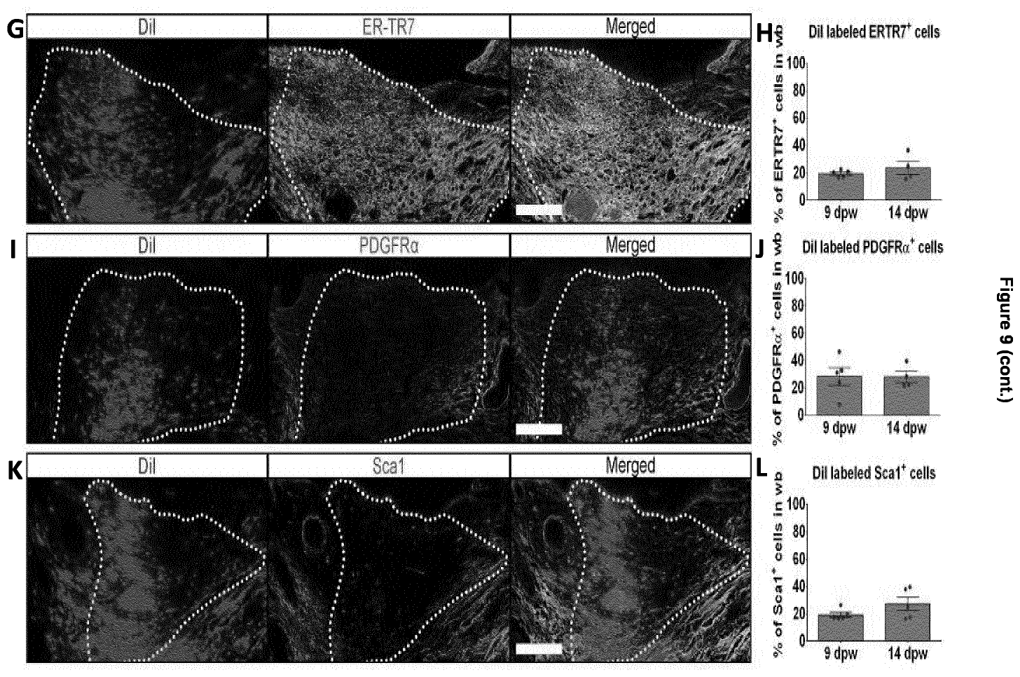

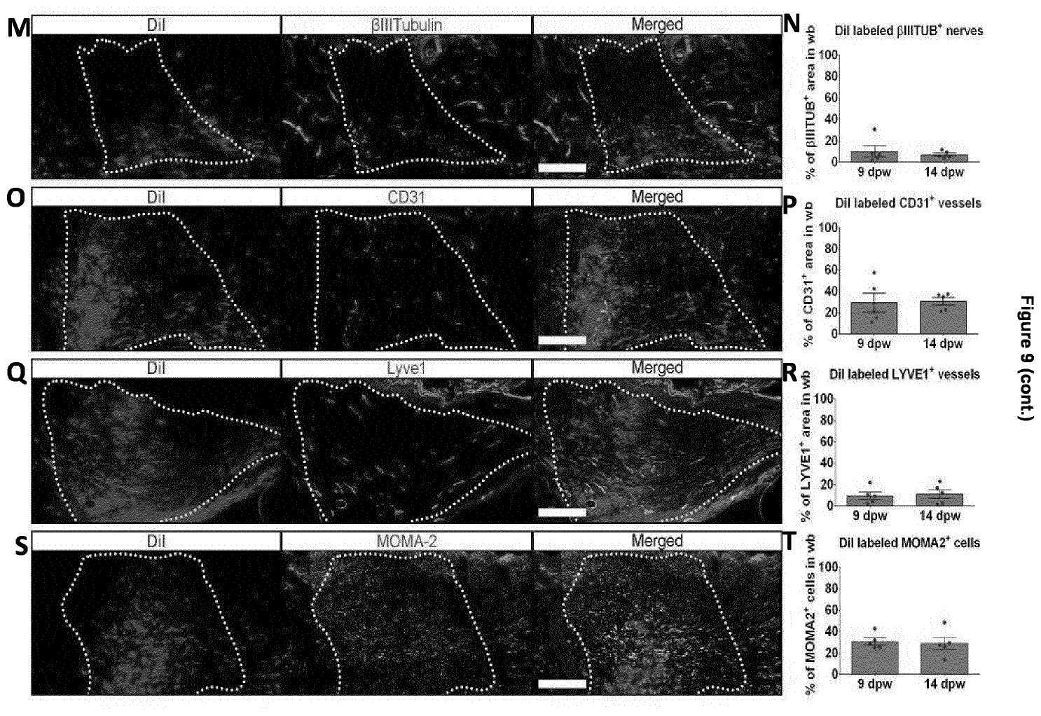

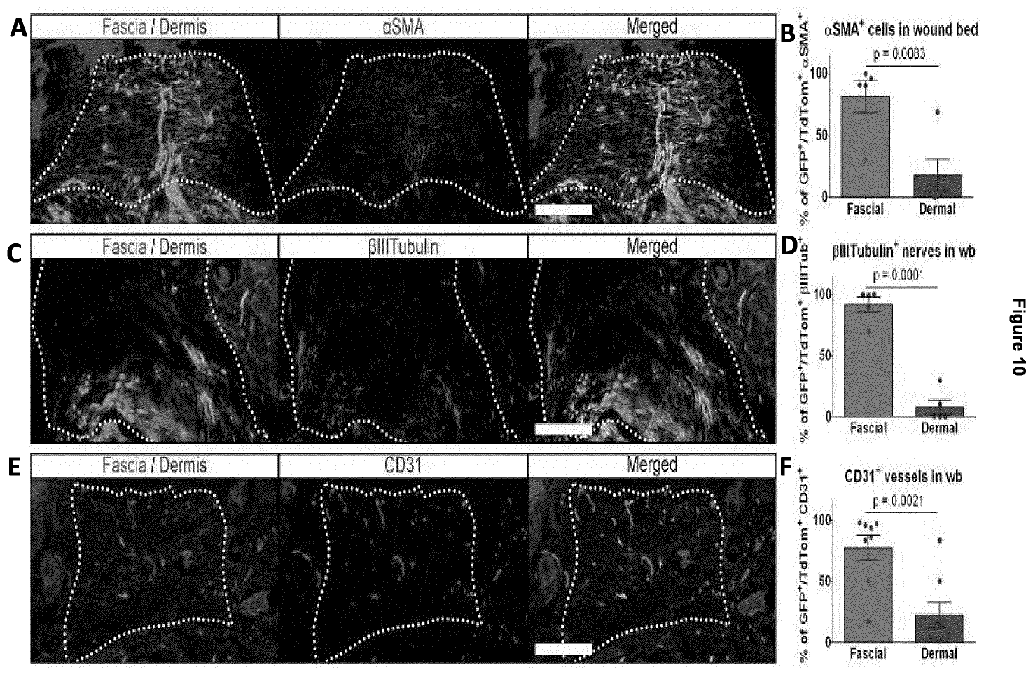

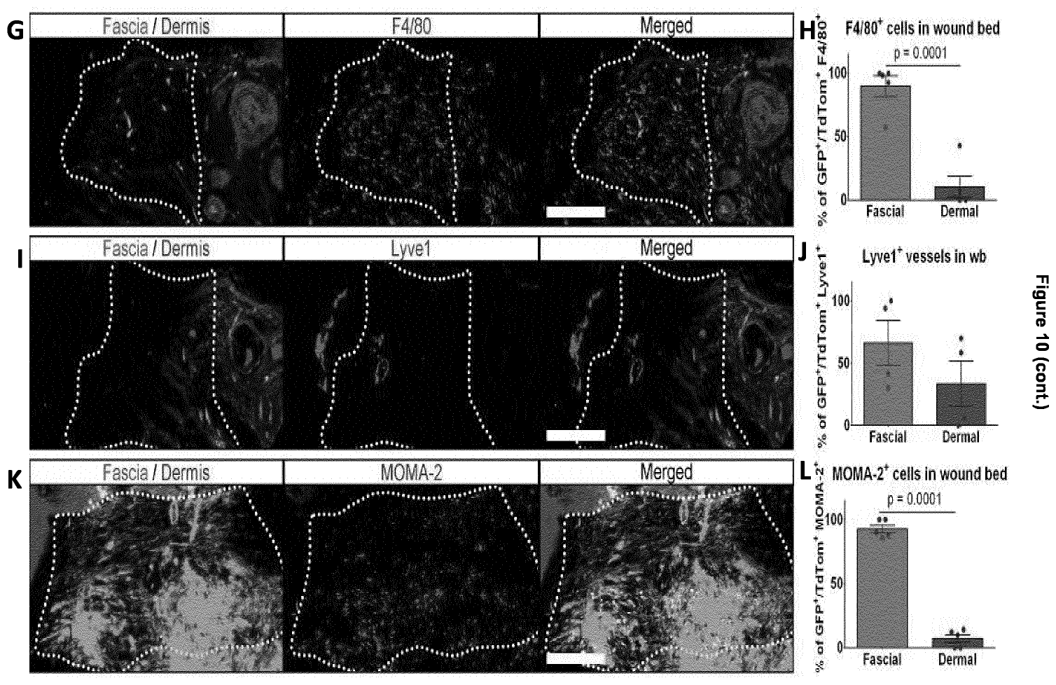

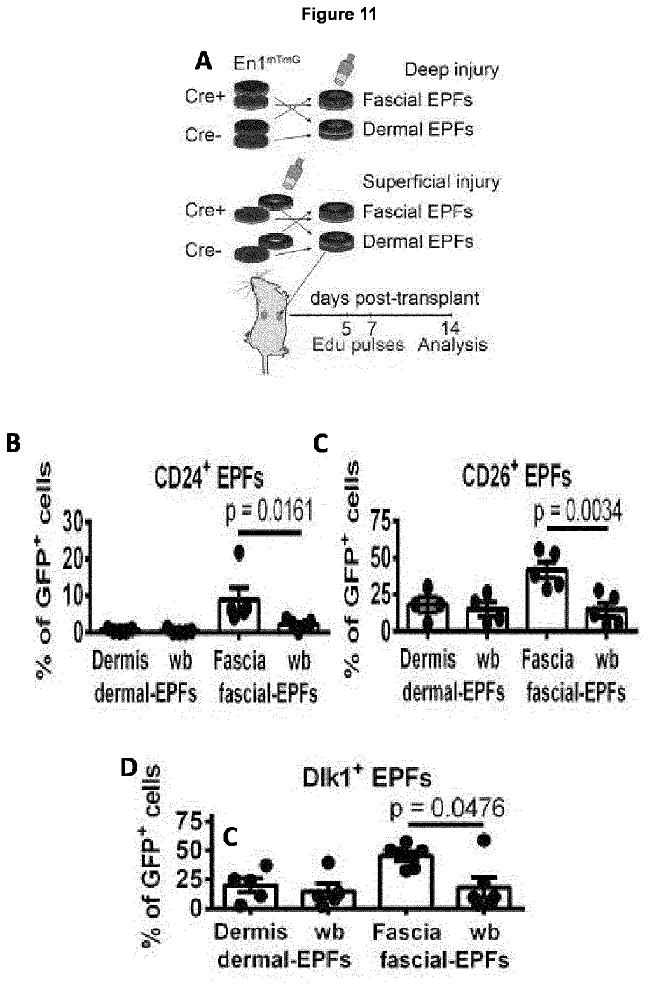

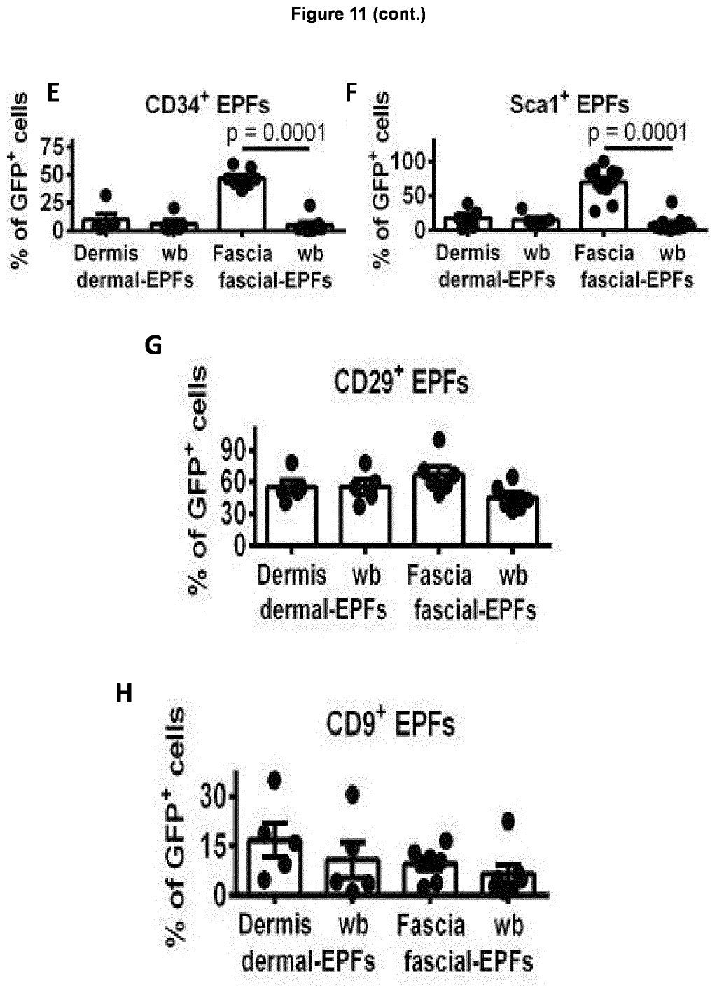

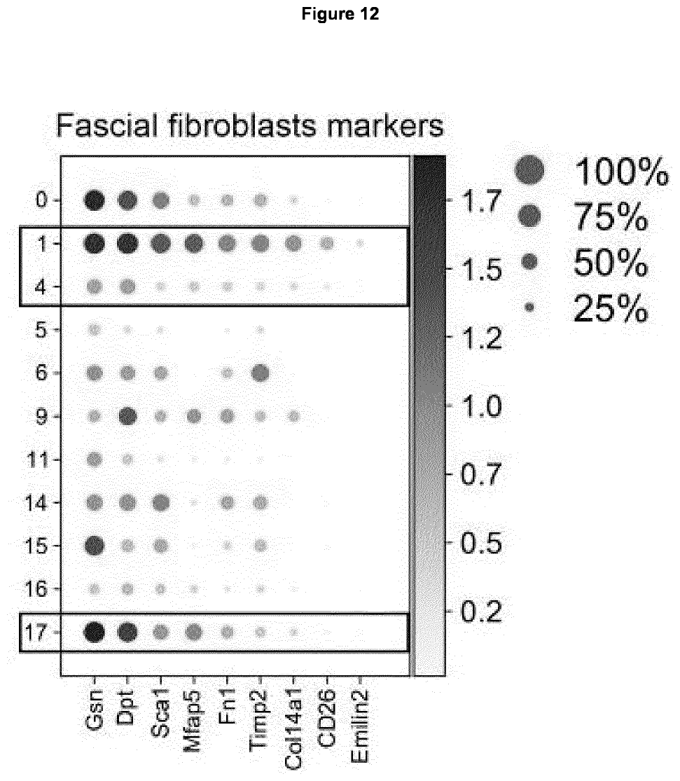

The hallmark of scar formation is the deposition of excessive collagen in the wound area which can be detected by histochemical staining. The skin tissues grown in the 96 well-plate under nutrient supplemented media showed gradual deposition of collagen within a fixed timespan (FIG. 1). Compared to scar formation on Day 0, the final scar formation on Day 5 had visually more collagen deposition (FIGS. 1G and H). The experiment was performed in triplicates and all produced the same results. Apart from the scar formation, a clear re-epithelialization process could also be observed in the D4 and D5 SCADs (FIG. 1E-H).

### Example 14: Scar Formation with SCAD in 384-Well Plate

The need to perform SCAD in higher throughput and on a larger scale was eminent. The previous experiment was performed in a 96-well plate. With over 30,000 chemical compounds, this would mean roughly 500 96-well plates. One way to tackle this was by trying to grow SCADs in a 384-well plate.

The SCADs grown in 384-well plates look morphologically different than those in 96-well plates. They look fragile and brittle and the scar and dermal tissue appeared to be deformed under microscope. Sectioning was performed using Tissue-Tek O.C.T freezed D5 SCAD samples collected from neonatal mice back-skin (see Example 1 and 2). Samples were stained using Masson's Trichrome staining (see Example 3).

All the images of tissue sections were taken under 10× magnifications with Axio Imager. The whole-mount images were taken with stereo microscope Leica M50 under 4× magnification (see Example 5).

The same process of scar development, when repeated in 384-well plates, fetched poor result as shown in FIG. 2. It was observed that in the 384-well plates the SCADs appeared brittle and hard visually and histological analysis showed inconsistent stain retention capacity and variability in data. The SCADs were difficult to section because of their brittleness. It can be assumed that the reduced quantity of media used in the 384-well plate (80 μl compared to 200 μl of 96 well plate) plays a role in the suboptimal development of the scar biopsies. Therefore, for the subsequent screening experiment, the 96-well format automation system was applied (FIG. 2F).

### Example 15: Normal Scar Formation in SCAD with Adult Human Back Skin

Further, scar development in D0-D5 mouse back-skin and human back-skin with the SCAD assay was monitored. For this experiment, the donor of back skin (human dorsal skin) was a healthy adult male after liposuction procedure. SCADs from human sample were prepared in a similar manner as applied for mouse back-skin and were grown in the same supplemented culture media as can be seen from Example 2.

However, the incubation days were extended to Day 7 and Day 10. The samples were sectioned and stained using Masson's Trichrome staining (see Example 3). The SCADs were fixed at D1, D2, D3, D4, D5, D7 and D10 to observe gradual structural changes associated with scar development. There was a gradual increase in tissue complexity and collagen deposition that was observed with time progression (FIG. 3) using Axio Imager (see Example 5).

### Example 16: Migration of Keratinocytes Observed in SCAD from Adult Human Back Skin

Skin wounds are repaired partially by the migration of keratinocytes to fill up the gap created by the wound. Epidermal keratinocytes can contribute to de novo hair follicle formation during the healing of larger wounds. Keratinocytes migrate with a rolling motion during the process of wound healing.

Here, the experiment is designed to test whether human back skin is capable of maintaining wound healing or scarring when cultured with the SCAD assay. This will serve to provide the foundation of the versatility of culture method.

SCADs were prepared as can be seen from Example 2, only that human dorsal adult back skin was used and the incubation days were extended to Day 7 and Day 10. The samples were sectioned and stained using Masson's Trichrome staining (see Example 3). The images were taken at 10× magnification using mosaic setting of Axio Imager (FIG. 4).

It was observed that from the timespan of Day 0 to Day 10, there is a gradual and continuous migration of epithelial keratinocytes (FIGS. 3 and 4) towards the ventral side of the tissue. This indicates two points—firstly, the SCAD assay is also applicable on human samples and secondly it demonstrates the movement/migration process of keratinocytes. The human skin however, is more structurally complex than the mouse back skin. With increasing number of days the tissues become more condensed and thick with collagen deposition which can be clearly observed in FIGS. 3 and 4. Using human and mouse skin supports the assumption that this assay may also be used for chemical trials (see Example 14).

### Example 17: Deposition of Matrix Fibers in D4 SCAD

Collagen, fibronectin and elastin are major matrix proteins found in the dermis of our skin. In developing SCADs, the amount of collagen-1, fibronectin and elastin present is monitored. For SCADs, it is important to demonstrate and quantify collagen-1 and fibronectin, as this could provide an insight about the excessive matrix deposited by fibroblasts.

SCAD tissues were prepared as described in Example 2 and immunostained for collagen 1 (Rockland 600-401-103), fibronectin (Abcam ab23750) and elastin (Abcam ab21610) in D4 SCADs (see Example 4). Representative 3D rendering images of D4 SCAD with immunolabelling of fibronectin, collagen I and elastin were generated (see Example 6), and samples were imaged using a laser scanning confocal microscope (see Example 5).

FIG. 5 shows the extracellular matrix fibre deposition in SCAD. The scar centres show extensive fibronectin fibres and less amount of mature collagen I fibres, and are virtually absent of elastin.

### Example 18: Marker Expression in SCAD

Cryosections of day 5 SCAD samples were prepared from wild type neonatal mouse back-skin (see Example 1 and 2). Then, those cryosection of day 5 SCAD samples were immunolabeled with anti-CK14 (Abcam ab181595), anti-CD26 (R&D systems AF954), anti-α-SMA (Abcam ab5694) and anti-N-Cadherin (Abcam ab18203) (see Example 4). Photomicrographs were documented by using a Zeiss AxioImager microscope with AxioVision software (Carl Zeiss) (FIG. 6A), or Zeiss laser scanning microscope LSM710 with Zen software (Carl Zeiss) (FIGS. 6B and C) (see Example 5).

These data show that at day 5, the keratinocytes migrate over the ventral side of biopsy, mimicking the “re-epithelialization” process of natural wound healing. The fibroblasts presented at the newly formed scar center express CD26, α-SMA and N-Cadherin, which can be used as markers to evaluate the scar formation (FIG. 6).

### Example 19: Chemical Screening of Nefopam with the SCAD Assay

The chemical Nefopam was selected (see Example 7) and further analyzed on SCAD (see Example 2). The treatment was done in triplicates.

When the SCAD samples were treated with the Prestw-229 (Nefopam hydrochloride), the samples had visually less scar formation in the whole-mount image. In the SCAD sections of treated samples, collagen formation was significantly less than observed in the control SCAD. Under Nefopam hydrochloride, there was visible reduction of scar development (FIG. 7).

### Example 20: Wound Healing Fibroblasts Come Primarily from Superficial Fascia

To test if deep fascia might contribute to wounding, specifically the deep fascia in living neonatal mice was labelled by localized subcutaneous injections of eGFP-expressing adenovirus (Ad-CMV-eGFP, see Example 8) near full-thickness excisional wounds (FIG. 8A). After 3 days there were multiple GFP⁺ cells derived from the deep fascia extending from the fascia below the wound up to the area directly beneath the scab (FIG. 8B).

Dil labeling of the deep fascia in excisional wounds of adult mice (FIG. 8C) resulted in the tagging throughout all the wound bed of 27.69±1.409% of the total cells at 9 days post-wounding (dpw) and 27.10±2.350% at 14-days post wounding, indicating a sustained presence of the fascial cells in the wound through the entire process (FIG. 8D-E).

Expression detection in Dil-labeled cells of fibroblast/mesenchymal markers (αSMA, CD29, CD90, ER-TR7, PDGFRα, and Sca1) indicates that classically defined fibroblasts in the wound bed originate from the fascia. In addition, nerves (βIIITubulin), endothelial (CD31), lymphatics (Lyve1), and macrophages (MOMA-2) markers expression in labeled cells showed that the deep fascia is an important contributor of all the major cell types present in the wound bed (Table 1 and FIG. 9).

TABLE 1
Oil label in marker positive cells*

| Marker | Cell type | 9 dpw | 14 dpw |
|---|---|---|---|
| αSMA | Myofibroblasts | 26.34 ± 3.557^{#} | 30.83 ± 10.75 |
| CD29 (Integrinβ1) | Fibroblasts | 23.77 ± 2.594 | 27.51 ± 4.760 |
| CD90 (Thy-1) | Fibroblasts | 29.34 ± 4.150 | 21.91 ± 5.353 |
| ER-TR7 | Fibroblasts | 19.48 ± 1.134 | 23.77 ± 4.712 |
| PDGFRα | Fibroblasts | 28.56 ± 6.310 | 28.17 ± 4.091 |
| Sca1 (Ly6a) | Fibroblasts/ | 19.17 ± 1.866 | 27.23 ± 4.939 |
| | Preadipocytes | | |
| βIIITubulin | Nerves | 10.35 ± 5.281 | 7.322 ± 1.476 |
| CD31 (Pecam-1) | Endothelial | 29.41 ± 8.865 | 30.57 ± 3.576 |
| Lyve1 | Lymphatics | 9.602 ± 3.437 | 11.29 ± 4.097 |
| MOMA-2 | Monocytes/ | 30.86 ± 3.286 | 29.30 ± 5.557 |
| | Macrophages | | |

*Percentage of total marker positive cells in the wound bed
^{#}Mean ± SEM

Next, a comprehensive fate mapping technique was demonstrated by transplanting skin grafts into living animals (see Example 12). Deep fascia was harvested from mice that constitutively express GFP and skin from mice that constitutively express TdTomato, and then placed the TdTomato⁺ skin over the GFP⁺ fascia. A full-thickness “wound” in the middle of the graft was made and then the entire chimeric tissue was transplanted into the back of adult mice (FIG. 8F). In these transplantation experiments, the entire cell contribution in host wounds could be observed from donor fascia or dermis, by analyzing the relative presence of GFP⁺ or TdTomato⁺ respectively (FIG. 8I). At 14 dpw, 80.04±3.443% of the labeled cells in the wound bed were GFP⁺, indicating they originated from the donor fascia. GFP label was seen uniformly throughout all skin layers from the deep fascia, through the hypodermis and dermal strata to underneath the newly regenerated epidermis that covered the wound (FIG. 8G-H).

Strikingly, fascial cells also populated the surrounding dermis. Cells from deep fascia (GFP⁺) made up 35.46±4.938% of the total labeled cells within a 0.2 mm radius around the wound (FIG. 8H-I). When analyzing the distribution of fascia-derived (GFP⁺) versus dermis-derived cells (TdTomato⁺) expressing specific cell type markers, consistently a higher contribution of the deep fascia to nerves (βIIITubulin⁺), blood vessel (CD31⁺), macrophages (F4/80⁺ or MOMA-2⁺), and myofibroblasts (αSMA⁺) in the wound bed was found. Only for lymphatics (Lyve1⁺) there was an equal contribution from dermis and deep fascia (Table 2 and FIG. 10). Collectively, these three independent fate-mapping approaches conclusively demonstrate that fascia is the major cellular source for all the cell types present in the wound bed.

TABLE 2
Cellular origin of marker positive cells*

| Marker | Cell type | Fascia | Dermis | p^{&} |
|---|---|---|---|---|
| αSMA | Myofibroblasts | 81.63 ± 12.84^{#} | 18.37 ± 12.84 | 0.0083 |
| βIIITubulin | Nerves | 91.89 ± 5.779 | 8.110 ± 5.779 | 0.0001 |
| CD31 | Endothelial | 77.53 ± 10.35 | 22.47 ± 10.35 | 0.0021 |
| (Pecam-1) | | | | |
| Lyve1 | Lymphatics | 66.42 ± 17.86 | 33.58 ± 17.86 | 0.2414 |
| MOMA-2 | Monocytes/ | 92.69 ± 3.059 | 7.306 ± 3.059 | 0.0001 |
| | Macrophages | | | |
| F4/80 | Monocytes/ | 89.62 ± 8.230 | 10.38 ± 8.230 | 0.0001 |
| | Macrophages | | | |

*Percentage of total GFP+ (Fascia)/TdTomato+ (dermis) and marker + cells in the wound bed at 14 days post-wounding
^{#}Mean ± SEM
^{&}Unpaired two-tailed t test, α = 0.05

### Example 21: Expression of Fibroblast Markers

It has already been shown that all scar-forming fibroblasts express Engrailed-1 (En1) early on in embryogenesis. and referring to these cells as En1-lineage positive fibroblasts or EPFs. Crossing the En1-Cre recombinase driver (En1^{Cre}) to a double-color fluorescent reporter (R26^{mTmG}), all EPFs across dermal and fascial compartments were lineage traced. Then chimeric skin transplants using these mice (En1^{Cre};R26^{mTmG}) were performed, to track the scar formation even more precisely by combining genetic lineage tracing with anatomic fate mapping. In these chimeric skin grafts, GFP⁺ label allows to trace the fates of the scar-forming EPFs from dermis or fascia. First fascia with traceable fibroblasts and untraceable dermis was used and then either a superficial wound through just the dermis and not the fascia below, or a full thickness excision through both tissues was made (FIG. 11A).

The expression of fibroblast markers was then examined, using the surrounding areas as controls to the wound bed fibroblasts. The adipocyte precursor marker CD9 and the general fibroblast marker CD29 were expressed in similar fractions of both dermal- and fascial-EPFs, and these fractions remained constant in the wound bed and control areas (FIG. 11G-H), indicating that the expression of these markers remain unaltered. As expected, hypodermal adipocytic (CD24 and CD34) and fibroblastic markers (CD26, Dlk1, and Sca1) were absent in dermal-EPFs in the control dermis, but prominent in fascial-EPFs in the normal fascia. Strikingly, the fraction of positive fascia-derived-EPFs in the wound bed dramatically decreased for all hypodermal markers (FIG. 11B-F) indicating that upon invasion into the wound bed, fascia-derived EPFs underregulate the expression of classical hypodermal markers.

Taken together, the results indicate that the migration extent of fascia-derived, and not dermis-derived fibroblasts, solely determines the scar severity.

1. A method for generating an ex vivo skin sample being capable of developing scar, comprising
(a) culturing a full thickness skin sample immersed and untethered in liquid culture;
(b) determining whether a scar is developed by the full thickness skin sample in step (a); and
(c) obtaining a scarred full thickness skin sample,
wherein the full thickness skin sample comprises fascia.

2. The method of claim 1, wherein the full thickness skin sample further comprises epidermis, dermis, subcutis.

3. The method of claim 1 or 2, wherein the full thickness skin sample is obtained from a mammal.

4. The method of any one of the preceding claims, wherein the full thickness skin sample is a punch biopsy.

5. The method of claim 4, wherein the punch biopsy is from a dorsal region.

6. The method of any one of claims 1 to 5, wherein the full thickness skin sample is a fresh sample.

7. The method of any one of claims 3 to 6, wherein the mammal is a mouse or human.

8. The method of claim 7, wherein the mouse is in a developmental fetal stage of at least E18.5 up to neonatal stage P10.

9. The method of any one of claims 1 to 8, wherein the full thickness skin sample has an average thickness of about 1 to 3 mm.

10. The method of any one of claims 1 to 9, wherein the liquid culture is suspension culture.

11. The method of any one of claims 1 to 10, wherein the full thickness skin sample is cultured for at least 4 days.

12. The method of any one of claims 1 to 11, wherein culturing is performed by using a DMEM/F-12 medium comprising 10% FBS, 1× GlutaMAX, 1× Penicillin/streptomycin, and 1× MEM non-essential amino acids.

13. The method of any one of claims 1 to 12, comprising determining whether the full thickness skin sample contains cells expressing CK14, Engrailed-1, CD26, N-Cadherin, alpha-smooth muscle actin (α-SMA), fibroblast specific proteins 1 (FSP1) and/or platelet derived growth factor receptors alpha (PDGFRα) and beta (PDGFRβ).

14. The method of any one of claims 1 to 12, comprising determining whether the full thickness skin sample contains cells expressing α-SMA, CD90, ER-TR7, PDGFRα, Sca1, βIIITubulin, CD31, MOMA-2, F4/80, CD24, CD34, CD26, Dlk1, Fn1, Col14a1, Emilin2, Gsn and/or Nov.

15. The method of any one of claims 1 to 12, wherein in step (b) determining whether a scar is developed by the full thickness skin sample in step (a) is done by visual inspection.

16. The method of any one of claims 1 to 12, wherein in step (b) determining whether a scar is developed by the full thickness skin sample in step (a) comprises determining whether collagen type I, collagen type III and/or fibronectin is present in said full thickness skin sample.

17. A method for screening for a compound which modulates scar development, comprising
(a) carrying out the method of any one of claims 1 to 16 in the presence of a compound of interest; and
(b) determining whether said compound of interest modulates scar development in comparison to carrying out the method of any one of claims 1 to 16 in the absence of said compound of interest.

18. The method of claim 17, wherein modulation of scar development is inhibition of scar development or promotion of scar development.

19. A preparation comprising a scarred full thickness skin sample obtainable by the method of any one of claims 1 to 16.

20. A preparation comprising a full thickness skin model comprising a full thickness skin sample immersed and untethered in liquid culture, wherein the full thickness skin sample comprises fascia.

21. The method of claim 20, wherein the full thickness skin sample further comprises epidermis, dermis, subcutis.

22. The preparation of claim 20 or 21, wherein the full thickness skin sample is obtained from a mammal.

23. The preparation of any one of claims 20 to 22, wherein the full thickness skin sample is a punch biopsy.

24. The preparation of claim 23, wherein the punch biopsy is from a dorsal region.

25. The preparation of any one of claims 20 to 24, wherein the full thickness skin sample is a fresh sample.

26. The preparation of any one of claims 22 to 25, wherein the mammal is a mouse or human.

27. The preparation of claim 26, wherein the mouse is in a developmental fetal stage of at least E18.5 up to neonatal stage P10.

28. The preparation of any one of claims 20 to 27, wherein the full thickness skin sample has an average thickness of about 1 to 3 mm.

29. The preparation of any one of claims 20 to 28 for use in a method for screening for a compound which modulates scar development.

30. The preparation of any one of claims 20 to 28 for use in therapy or diagnosis.
